(19) United States
(12) Patent Application Publication
(10) Pub. No.: US 2024/0087764 A1
(43) Pub. Date: Mar. 14, 2024

(54) LEAD-CONTAINING FLEXIBLE RADIATION-PROTECTIVE COMPOSITIONS AND PROTECTIVE ARTICLES

(71) Applicant: BURLINGTON MEDICAL, LLC, Newport News, VA (US)

(72) Inventors: Anita T. Broach, Christiansburg, VA (US); David Arnold, Newport News, VA (US); Eric Cornavaca, Newport News, VA (US); Lee Ann Fachko, Newport News, VA (US)

(21) Appl. No.: 18/134,243

(22) Filed: Apr. 13, 2023

Related U.S. Application Data
(60) Provisional application No. 63/330,487, filed on Apr. 13, 2022.
(60) Provisional application No. 63/457,337, filed on Apr. 5, 2023.

(51) Int. Cl. G21F 1/10; G21F 1/12

(52) U.S. Cl. CPC G21F 1/106; G21F 1/125

(57) ABSTRACT

Certain embodiments are described that are directed to lead containing compositions including radiation absorption metals in combination with a polymeric material. In some aspects, a composition includes lead, optionally at least one additional heavy metal known to have shielding capability against ionizing radiation and at least one polymer, polymer blend or co-polymer. Sheets and articles including the compositions are also described.

### PRIORITY APPLICATION

This application is related to, and claims priority to and the benefit of, each of U.S. Provisional Application No. 63/330,487 filed on Apr. 13, 2022 and U.S. Provisional Application No. 63/457,337 filed on Apr. 5, 2023, the entire disclosure of each of which is hereby incorporated herein by reference for all purposes.

### TECHNOLOGICAL FIELD

Certain configurations are described of lead-containing flexible radiation-protective compositions that can be used in protective articles and other articles to protect against radiation such as, for example, primary X-rays and scattered X-rays.

### BACKGROUND

X-rays are often used in various medical imaging procedures. Various shielding articles are often used when a patient is subjected to the X-rays.

### SUMMARY

Certain configurations, illustrations, examples and embodiments described herein relate to light-weight, lead-containing flexible, abrasion- and crack-resistant metal-filled compositions with improved X-ray shielding capability over a broad range of X-ray energies ranging from 10 kV to about 140 kV. Embodiments of the compositions can provide protection against direct X-rays (primary X-ray radiation) and scatter X-rays, including secondary and tertiary X-rays. Furthermore, methods of making such compositions, methods of making single-layer and multi-layer radiation-protective sheet materials using the metal-filled compositions, and methods of making radiation-protective articles are also described. In some embodiments, the protective articles include, but are not limited to, aprons, vests, gloves, shields, skirts, sleeves, drapes, pads, curtains, sheets and other protective garments and radiation-shielding elements.

In an aspect, a radiation-shielding metal-filled elastomeric composition comprises lead and at least one metal selected from heavy metals known to have shielding capability against ionizing radiation, and having atomic (Z) number of at least 50, at least one polymer, polymer blend or co-polymer, selected from the group of polymers known to yield elastomeric materials with pronounced visco-elastic properties, and additives, such as plasticizers, wetting agents, flow modifiers and others, where the fraction of the metals is in the range from 50 wt % to about 90 wt % based on the total weight of the composition.

In another aspect, a radiation-shielding metal-filled elastomeric composition comprises a mixture of lead and at least one heavy metal having a shielding capability against ionizing radiation, wherein the at least one heavy metal has an atomic (Z) number greater than or equal to 50, at least one matrix comprising a polymer, polymer blend or co-polymer providing an elastomeric material with pronounced visco-elastic properties, and additives, such as plasticizers, wetting agents, flow modifiers and others.

In an additional aspect, a radiation-shielding metal-filled elastomeric composition comprises a mixture of lead and at least one heavy metal having a shielding capability against ionizing radiation, wherein the at least one heavy metal has an atomic (Z) number greater than or equal to 50, at least one matrix comprising a polymer, polymer blend or co-polymer providing an elastomeric material with pronounced visco-elastic properties, and additives, such as plasticizers, wetting agents, flow modifiers and others.

In another aspect, a radiation-shielding metal-filled elastomeric composition comprises lead and at least one metal, different than lead, having an atomic (Z) number of at least 50, wherein the metal is present from 50 wt % to about 90 wt % in the composition to absorb radiation, and at least one polymer, polymer blend or co-polymer selected from the group consisting of polymers known to yield elastomeric materials with pronounced visco-elastic properties, and optionally one or more additives, including plasticizers, wetting agents, flow modifiers and others. For example, the polymer can be one or more of polyvinyl chloride (PVC) elastomer (plastisol), polyolefin elastomer, natural rubber, a synthetic rubber, a urethane-type elastomer, a silicone-type elastomer, a blend of at least two different types of polymers, a copolymer or a polymer with at least two different monomer repeating units.

In some examples, the metal is a heavy metal and has atomic (Z) number of 50 or greater. In some instances, the metal is a mixture lead and at least two different heavy metals with Z-numbers of at least 50. In some embodiments, the metals used are in a form of metal alloy. In other embodiments, the metal is used in its pure elemental form. In additional embodiments, the metal is used as a compound, such as carbonate, sulfate, oxide, or a combination of different metal forms. The metal materials may be layered individually or any one layer may comprise two or more different metals as noted herein.

In certain embodiments, the at least one polymer, polymer blend or co-polymer is present in a fiber. In some examples, the heavy metal is coated or otherwise disposed onto or into the fiber. In other examples, the fiber comprises one or more of polyethylene (PE), polypropylene (PP), polyester terephthalate (PET), polyethylene naphthalate (PEN), nylon, polyacrylonitrile (PAN), polyamide, polycarbonate (PC), aramid, or combinations thereof. In additional examples, the heavy metal is distributed in the at least one polymer, polymer blend or co-polymer in a substantially uniform distribution. In other embodiments, the heavy metal is distributed in the at least one polymer, polymer blend or co-polymer in a gradient distribution. In some embodiments, a mixture of the metal and the polymer can be prepared and fibers containing the combined materials can be formed, e.g., by extrusion, spinning, drawing, etc.

In some embodiments, the metal is a mixture of lead and at least one heavy metal and wherein one of the metals of the mixture is present in a non-uniform distribution in the polymer, polymer blend or co-polymer. For example, a gradient distribution of one or both of the heavy metals can be present.

In another aspect, a radiation-shielding material sheet comprises at least one layer comprising a metal-filled elastomer composition as described herein, and additional layers adding additional functionality to the radiation-shielding material. In certain embodiments, the material can be a single layer filled with lead and at least one heavy metal. In other embodiments, the material includes (or consists of) of two different metal-filled elastomer layers, at least one of which contains lead and at least one heavy metal filler. In some embodiments, the material includes (or consists of) more than two elastomer layers, at least one of which contains lead and at least one heavy metal filler. In other examples, the material includes (or consists of) at least one elastomer layer and at least one layer from a different material class, such as fabric.

In another aspect, an apron comprising one of more of the compositions as described herein is disclosed. In another aspect, a vest comprising one of more of the compositions as described herein is disclosed. In another aspect, a glove comprising one of more of the compositions as described herein is disclosed. In another aspect, a skirt comprising one of more of the compositions as described herein is disclosed. In another aspect, a sleeve comprising one of more of the compositions as described herein is disclosed. In another aspect, a drape comprising one of more of the compositions as described herein is disclosed. In an additional aspect, a pad comprising one of more of the compositions as described herein is disclosed. In another aspect, a curtain comprising one of more of the compositions as described herein is disclosed. In another aspect, a protective garment comprising one of more of the compositions as described herein is disclosed. In another aspect, a blanket comprising one of more of the compositions as described herein is disclosed. In another aspect, a thyroid shield comprising one of more of the compositions as described herein is disclosed. In another aspect, a protective wrap comprising one or more of the compositions as described herein is disclosed. In another aspect, a protective cap comprising one or more of the compositions as described herein is disclosed.

In another aspect, a lead containing radiation-shielding metal-filled elastomeric composition comprises lead and at least one metal selected from heavy metals known to have shielding capability against ionizing radiation and having atomic (Z) number of at least 50. The composition can also include a matrix comprising at least one polymer, polymer blend or co-polymer selected from the group consisting of thermoplastic polymers, thermoset polymers, elastomeric polymers, visco-elastic polymers, and combinations thereof. The lead and the at least one metal are together in the range from 50 wt % to about 90 wt % based on the weight of the lead containing radiation-shielding metal-filled elastomeric composition, and wherein the lead and the at least one metal are arranged in the matrix to provide protection from both primary X-rays and scatter X-rays.

In certain embodiments, the at least one polymer, polymer blend or co-polymer is polyvinyl chloride (PVC) elastomer, and wherein the composition further comprises a plasticizer. In other embodiments, the at least one polymer, polymer blend or co-polymer is selected from the group consisting of a polyolefin elastomer, a natural rubber, a synthetic rubber, a urethane-type elastomer, a silicone-type elastomer, a vinyl acetate polymer, a vinyl chloride polymer, an ethylene-vinyl hexyl copolymer, an ethylene-vinyl acetate copolymer, a blend of at least two different types of polymers, and a co-polymer comprising at least two different monomer repeating units.

In some embodiments, the metal is a mixture of at least two different heavy metals each with Z-numbers of at least 50. In other embodiments, the metals used are in a form of metal alloy or used in its pure elemental form or a metal carbonate, a metal sulfate, a metal oxide, or a combination of different metal forms.

In certain embodiments, the at least one polymer, polymer blend or co-polymer is present in a fiber. In some embodiments, the heavy metal is coated into the fiber. In some configurations, the fiber comprises one or more of polyethylene (PE), polypropylene (PP), polyester terephthalate (PET), polyethylene naphthalate (PEN), nylon, polyacrylonitrile (PAN), polyamide, polycarbonate (PC), aramid, or combinations thereof.

In some embodiments, the heavy metal is distributed in the at least one polymer, polymer blend or co-polymer in a substantially uniform distribution or in a gradient distribution. In other embodiments, the metal is a mixture of two different heavy metals and wherein one of the heavy metals of the mixture is present in a non-uniform distribution in the polymer, polymer blend or co-polymer.

In certain embodiments, the heavy metals include Bi in combination with one or more of Sb, W, Sn or Ba, wherein the Bi is present in an amount that exceeds the amount of each of the Sb, W, Sn or Ba.

In other embodiments, the heavy metals include Sb in combination with one or more of Bi, W, Sn or Ba, wherein the Sb is present in an amount that exceeds an amount of each of the Bi, W, Sn or Ba.

In some embodiments, the heavy metals include W in combination with one or more of Bi, Sb, Sn or Ba, wherein the W is present in an amount that exceeds an amount of each of the Bi, Sb, Sn or Ba.

In other embodiments, the heavy metals include Ba in combination with one or more of Bi, Sb, W or Sn, wherein the Ba is present in an amount that exceeds an amount of each of the Bi, Sb, W or Sn.

In certain embodiments, the at least one metal is present in the composition as particles, and wherein the particles have different sizes including micron size particles and nanosize particles.

In another aspect, a radiation-shielding material sheet comprising comprises a lead containing radiation-shielding metal-filled elastomeric composition as described herein. In certain embodiments, the sheet comprises a first layer and a second layer, and wherein the lead containing radiation-shielding metal-filled elastomeric composition is present in the first layer. In other embodiments, the sheet is a single layer comprising the lead containing radiation-shielding metal-filled elastomeric composition. In some embodiments, the sheet comprises a first layer and a second layer, wherein the lead containing radiation-shielding metal-filled elastomeric composition is present in the first layer, and wherein the second layer comprises an additional lead containing radiation-shielding metal-filled elastomeric composition comprising at least one meta selected from heavy metals known to have shielding capability against ionizing radiation, and having atomic (Z) number of at least 50, at least one polymer, polymer blend or co-polymer selected from the group consisting of elastomers, visco-elastic polymers, and additives, such as plasticizers, wetting agents, flow modifiers and others, wherein the at least one metal in the additional lead containing radiation-shielding metal-filled elastomeric composition is in the range from 50 wt % to about 90 wt % based on the weight of the additional lead containing radiation-shielding metal-filled elastomeric composition, and wherein the at least one metal is arranged in the matrix of the additional lead containing radiation-shielding metal-filled elastomeric composition to provide protection from both primary X-rays and scatter X-rays. In other embodiments, the sheet comprises a first layer and a second layer, and wherein the second layer comprises a fabric.

In another aspect, an article comprises a lead containing radiation-shielding metal-filled elastomeric composition as described herein. For example, the article can be selected from the group consisting of an apron, a vest, a glove, a skirt, a sleeve, a drape, a pad, a curtain, a blanket, a thyroid shield and a protective wrap. In some embodiments, the article comprises a fabric layer comprising the lead containing radiation-shielding metal-filled elastomeric composition. In other embodiments, the article comprises a fabric layer an additional layer coupled to the fabric layer, wherein the additional layer comprises the lead containing radiation-shielding metal-filled elastomeric composition.

In another aspect, a pellet comprises a lead containing radiation-shielding metal-filled elastomeric composition as described herein.

Additional aspects, embodiments, configurations and features are described in more detail below.

### BRIEF DESCRIPTION OF THE SEVERAL VIEWS OF THE DRAWINGS

The accompanying drawings illustrate certain aspects, embodiments features and elements of and should not be used to limit the scope of the appended claims. Together with the written descriptions, the drawings and embodiments serve to explain certain principles and illustrations to facilitate a better understanding of the compositions, articles and processes described herein.

FIG. 1 is a flowchart of making X-ray protective articles starting with mixing/compounding/blending of the raw materials for making flexible X-ray protective material (sheets), which then are cut and tailored into final X-ray protective articles.

FIG. 2 is a schematic presentation of attenuation of incident X-rays passing through a metal-filled material.

FIG. 3 is a schematic presentation of a cross-section of an elastomer matrix filled with protective metal particles, and various additives such as but not limited to wetting agents, reinforcing fibers, flow modifiers, rheology modifiers, binders, etc.

FIG. 4 is a schematic presentation of a cross-section of an elastomer matrix filled with protective metal particles with a particular particle size distribution.

FIG. 5 is a schematic presentation of a cross-section of an elastomer matrix filled with two types of protective metal particles, particles with high atomic (Z) number and particles with low atomic (Z) number.

FIG. 6 is a schematic presentation of a cross-section of a radiation protective material comprising two layers, a thicker layer made of an elastomer filled with metal particles of low atomic (Z) number and higher mean particle sizes, and a thinner layer made of elastomer filled with metal particles of low atomic (Z) number and lower mean particle sizes.

FIG. 7 is a schematic presentation of a cross-section of radiation protective material comprising two layers, a thicker layer made of an elastomer filled with metal particles of high atomic (Z) number and lower mean particle sizes, and a thinner layer made of elastomer filled with metal particles of low atomic (Z) number and higher mean particle sizes.

FIG. 8 is a schematic presentation of a cross-section of radiation protective material comprising two layers, a thicker layer made of an elastomer filled with metal particles of high atomic (Z) number and a layer made of elastomer filled with metal particles of low atomic (Z) number, both layers made of metal particles with wide particle size distributions.

FIG. 9 is a schematic presentation of a cross-section of radiation protective material comprising two layers, where one of the layers comprises two types of metal particles of low atomic (Z) number and medium atomic (Z) number, and the second layer comprises metal particles of high atomic (Z) number.

FIG. 10 is a schematic presentation of a cross-section of radiation protective material comprising two layers, each comprising a metal of different Z-number, and the two layers are physically-separated (distinct) layers.

FIG. 11 is a schematic presentation of a cross-section of radiation protective material comprising two layers, each comprising a metal of different Z-number, and the two distinct layers are hold together by a thin adhesive layer.

FIG. 12, FIG. 13, FIG. 14 and FIG. 15 are schematic presentations of different types of fabrics, knitted and woven ones, used in the present invention.

FIG. 16 is a schematic presentation of a cross-section of a hybrid radiation protective material comprising two different layers, a fabric and an elastomer layer, one or both comprising protective metal particles.

FIG. 17 is a schematic presentation of a cross-section of a hybrid radiation protective material comprising two different types of layers, a fabric embedded in elastomer layers, where at least one of them comprises protective metal particles.

FIG. 18 is a schematic presentation of a cross-section of a hybrid radiation protective material comprising an elastomer layer sandwiched between two fabric layers, where at least one of the layers is comprising protective metal particles.

FIG. 19 is a schematic presentation of a cross-section of a hybrid radiation protective material comprising three-layer structure, a fabric embedded between a thermoset elastomer layer and a thermoplastic elastomer layer, where at least one of the layers contains protective metal particles.

FIG. 20 is a schematic presentation of a cross-section of a hybrid radiation protective material comprising three-layer structure, a fabric embedded between an elastomer layer and a temporarily (PVA) layer, where at least one of the layers contains protective metal particles.

FIG. 21 is a schematic of a cross-section of a fabric coated with a coating containing protective metal particles on one side, where the metal type is chosen from a category of radiation protective metals.

FIG. 22 is a schematic of a cross-section of a fabric coated with a coating containing protective metal particles on both sides, where the metal type is chosen from a category of radiation protective metals.

FIG. 23 is a schematic of a cross-section of a fabric coated with a coating containing protective metal particles on both sides, where the metal type is chosen from a category of radiation protective metals. The elastomer layer is added to enhance the flex properties of the material.

FIG. 24 is a schematic of a cross-section of a fabric coated with a coating containing protective metal particles on both sides, where the metal type is chosen from a category of radiation protective metals. The elastomer layers on both sides of the fabric are added to enhance the flex properties of the material.

FIG. 25 is a schematic of a cross-section of a fabric coated with a continuous metal layer on one side, where the metal type is chosen from a category of radiation protective metals.

FIG. 26 is a schematic of a cross-section of a fabric coated on both sides with continuous metal layers, where the metal type is chosen from a category of radiation protective metals.

FIG. 27 is a schematic of a cross-section of a fabric coated with a continuous metal layer, where the metal type is chosen from a category of radiation protective metals, and an elastomer layer is added to enhance the flex properties of the material and protect the metal layer.

FIG. 28 is a schematic of a cross-section of a fabric coated with a continuous metal layer, where the metal type is chosen from a category of radiation protective metals, and elastomer layers are added to enhance the flex properties of the material and protect the metal layers.

FIG. 29 is a schematic presentation of making radiation protective gloves by immersing them in a tank with aqueous solution of PVA comprising metal protective particles, which upon drying are ready to use. After use, the gloves are disposed in a tank with warm water, where the protective metal particles are being regenerated and the gloves are disposed.

FIG. 30 is a drawing of the primary and scatter X-rays generated during X-ray diagnostic or interventional procedure. The primary X-rays passing through the patient are being scatter in all directions, thus generating secondary and tertiary X-rays.

FIG. 31, FIG. 32, FIG. 33 and FIG. 34 show an illustrative frontal X-ray protective apron.

FIG. 35, FIG. 36 and FIG. 37 show an illustrative X-ray protective vests.

FIG. 38 shows a drawing of an illustrative X-ray protective skirt.

FIG. 39 shows a drawing of an illustrative leg X-ray protective wrap.

FIG. 40, FIG. 41 and FIG. 42 show an illustrative X-ray protective body wraps.

FIG. 43 shows an illustrative X-ray protective cap.

FIG. 44 shows an illustrative arm X-ray protective sleeve.

FIG. 45, FIG. 46 and FIG. 47 show illustrative X-ray protective thyroid shields.

FIG. 48 shows an illustration of micron size particles in combination with an elastomer.

FIG. 49 shows an illustration of nanosize particles in combination with an elastomer.

FIG. 50 shows an illustration of an elastomer with different building blocks.

FIG. 51 shows an illustration of a matrix with liquid crystals.

FIG. 52 and FIG. 53 show a matrix with an interpenetrating network.

FIG. 54 is an illustration showing a functional coating on a shielding layer.

FIG. 55 is an illustration showing an additional layer on an X-ray protection layer.

FIG. 56 is an illustration showing an additional layer on an X-ray protection layer.

FIG. 57 is an illustration showing a thin metal layer on an X-ray protection layer.

FIG. 58 is an illustration showing a vest with areas of different absorption materials.

FIG. 59 is a skirt showing areas of different absorption materials.

FIG. 60 is an illustration showing an X-ray shielding apron with areas of different absorption materials.

FIG. 61 is an SEM image showing tungsten particles.

FIG. 62 is an SEM image showing antimony particles.

FIG. 63 is an SEM image showing bismuth particles.

FIG. 64 is an SEM image showing bismuth oxide (B₂O₃) particles.

FIG. 65 is an SEM image showing tin particles.

FIG. 66 is an SEM image showing barite (BaSO₄) particles.

FIG. 67, FIG. 68 and FIG. 69 show mechanical properties of certain copolymer materials.

FIG. 70 is a graph showing tensile properties of lead-polyolefin compositions.

FIG. 71 is a graph showing tear strength of lead-polyolefin compositions.

FIG. 72 and FIG. 73 are attenuation values measured using BBG* and NBG modes, respectively.

FIG. 74 and FIG. 75 are tables showing percentage attenuation at different source voltages for BBG* and NBG modes, respectively.

FIG. 76 and FIG. 77 are tables showing attenuation ratios at different source voltages for BBG* and NBG modes, respectively.

FIG. 78 and FIG. 79 are graphs showing calculated LEV values at different source voltages for BBG* and NBG modes, respectively.

FIG. 80 is a graph showing tensile properties of Pb—Sb compositions.

FIG. 81 is a graph showing tear strength of Pb—Sb compositions.

FIG. 82 is a graph showing tensile properties of Pb—Sb—Ba compositions.

FIG. 83 is a graph showing tensile properties of Pb—Sb—W—Ba compositions.

FIG. 84 and FIG. 85 are tables showing percentage attenuation and LEV Values at different source voltages for BBG* measurements of Pb—Sb compositions, respectively.

FIG. 86 and FIG. 87 are tables showing percentage attenuation and LEV Values at different source voltages for NBG measurements of Pb—Sb compositions, respectively.

FIG. 88 and FIG. 89 are tables showing attenuation ratios at different source voltages for BBG* and NBG modes, respectively.

FIG. 90 and FIG. 91 are graphs showing calculated LEV values at different source voltages for BBG* and NBG modes for Pb—Sb compositions, respectively.

FIG. 92 is a graph showing tensile strength for Pb—Sb compositions including recycled content.

FIG. 93 is a graph showing tear strength for Pb—Sb compositions including recycled content.

FIG. 94 is a table showing attenuation percentages of Pb—Sb compositions including recycled content.

FIG. 95 is a table showing attenuation ratios of different lead and non-lead compositions.

The illustrations shown in the figures are intended to facilitate a better understanding of certain aspect, embodiments and features and are not intended to be limiting or necessarily descriptive of every aspect, embodiment or feature.

### DETAILED DESCRIPTION

The aspects, embodiments and examples described herein are related to lead-containing compositions with improved X-ray shielding properties, methods of making such compositions, methods of making the protective material sheets using the disclosed compositions, as well as methods of making the protective articles, such as but not limited to garments, vests, aprons, skirts, shields, curtains, pads, gloves, blankets, sleeves, etc.

In certain embodiments, while the exact materials in any composition may vary, the compositions generally include lead, optionally in combination with one or more other heavy metals, and an elastomeric matrix. For example, different elastomer classes and their combinations, e.g., polyolefins, vinyls, silicones, natural rubbers, etc., can be used in combination with the metal particles. In some embodiments, the matrix may be plastisol, which can be extruded into desired shapes, can be pelletized or otherwise can be processed to provide final articles. Plastisol generally includes PVC in combination with plasticizers. The plastisol can be heated in the presence of the metals and other additives, which generally causes the plastic particles to absorb the plasticizer and form a viscous gel trapping the metals and other additives. Once the product is cooled it becomes a flexible plasticized product. The resulting material can be formed into sheets, articles, fibers or other arrangements.

The compositions described herein can include different metal combinations and metal particle sizes. For example, the smaller particles may be better at occupying more volume, which can result in lighter products and superior X ray protection. If desired, the compositions can include a mixture of larger, mid-size, and small particles, e.g., a mix of particles with 50-300 microns, 10-100 microns, and less than 5 microns.

Certain configurations of the lead-containing compositions described herein are designed to provide protection against direct X-rays (primary X-ray radiation) and scatter X-rays, including secondary and tertiary X-rays. These effects can be achieved in numerous ways using numerous different materials. For example, metals with different particle sizes and/or distributions can be present in one or more layers comprising a polymer or other material to provide an arrangement that can absorb or attenuate direct X-rays and scatter X-rays. In some instances, two metals with different particle sizes and/or different gradient distributions can be present to enhance the attenuation effects further. While the exact composition may vary depending on the intended use of the radiation protective materials, the composition typically includes lead and at least one metal with an atomic number Z greater than 50 that is used in combination with an elastomeric matrix which can retain the metal(s) and permits stretching without tearing or breaking of the materials Illustrative amounts of the materials which can be present in the compositions include a total metal content of about 40 wt % to about 90 wt %, polymer content of about 10 wt % to about 60 wt % and additional materials which can be present up to 50 wt %, e.g., plasticizers, fillers, etc. The compositions can provide protections over a broad X-ray energy range, e.g., 50 keV to 150 keV.

The compositions can generally be used to prepare final articles. For example, fibrous sheets can be produced by weaving, knitting, braiding fibers or any other method known in the art. Then the fibrous sheets are coated, sprayed, impregnated, or being deposited with metal particles or metal-comprising coatings, where at least one metal is radiation-protective metal. In some configurations, the fibrous protective sheets are made of metal-comprising fibers, bundles of fibers, yarns, threads or filaments, where at least one of the metals embedded n the fibers, filaments, yarn or thread is radiation protective metal. In some embodiments, such metal-comprising fibers, bundles of fibers, filaments, threads or yarns can be made solely of metal, or in other embodiments can be made of hybrid materials, i.e. made of other-than-metal material, organic (polymer, composite) or inorganic (ceramic), and a metal. In yet another embodiment, fibers, bundles of fibers, filaments, threads or yarns, are made of organic material (polymer), and they sprayed, coated, or impregnated with metal particles and or coating comprising metal particles. In other embodiments, bulk hybrid materials comprising metal particles or metal fibers and a polymer are used to produce the fibers, bundles of fibers, filaments, threads or yarns. As example only, such material can be virgin or already used and disposed from the radiation protection industry, hospitals etc. Such material can be shredded to make shapes (granules, pellets) that will be used for making the fibers, bundles of fibers, filaments, threads or yarns. If desired, the articles made of the radiation protective materials can include components such as sensors and/or detectors to sense and/or detect, for instance, radiation level exposure, UV exposure, temperature, pressure, and or other parameters of interest. There may be nanosize particles (either alone or in combination with micron size particles or particles of other size) to enhance attenuation of different types of X-rays.

Interventional procedures using ionizing radiation have revolutionized the medicine in the past decade. Physicians in different medical and surgical fields, assisted by nurses and radiological technologists, perform interventions guided by radiological imaging. In general, these interventions are less invasive, the lesions that were not previously accessible, can now be treated with the image-guided interventions. Furthermore, such interventions are better suited for patients who may not tolerate anesthesia, as well as the recovery periods are shorter and the complication rates are lower than those for the equivalent conventional surgeries. However, the extensive use of X-ray imaging for diagnosis and/or radiation-guided interventional procedures raise the issue of occupational health hazards for the health care workers working in medical settings, where X-rays are often used. For example, mammography often used a tube accelerating voltage of 20-30 kV and an average photon energy of 20 keV. Dental diagnostics often use a tube accelerating voltage of 60 kV and an average photon energy of 30 keV. General diagnostics often use a tube accelerating voltage of 40-140 kV and an average photon energy of 40 keV. CT imaging often uses a tube accelerating voltage of 80-140 kV and an average photon energy of 60 keV. The radiation exposure of the physicians, nurses and technologists working in such environments accumulated over many years have been associated with adverse health effects, among which are eye disorders (cataracts), risks of cancer induction (brain, breast, bone and skin cancers, leukemia, lymphoma), thyroid issues, hypertension, atherosclerosis, etc. Musculoskeletal problems, including those of the spine and orthopedic issues in health care professionals working in radiation settings have been reported and have been linked to the cumulative burden of wearing heavy protective aprons and other protective garments.

In recent years, scatter X-ray radiation originating from the primary X-rays being scattered from the patient and the surrounding objects in clinical practices have been identified as a big threat for the medical staff. The scatter X-ray radiation usually has lower photon energy and it affects the operators from different angles, meaning longer path lengths for the rays approaching the operators at angles different than normal incidence. For instance, it is known that during longer medical procedures, such as coronary interventions, peripheral vascular interventions, heart catheter, angiography, etc., the radiation dose received by physicians and the staff is almost entirely attributable to the radiation scattered from the patients and the surrounding. The length of the procedures and the repeated exposure to such even low dose of low energy X-rays over many years is concerning for the health and wellbeing of the medical staff involved.

X-rays are typically scattered by what is referred to as the Compton effect. This incoherent scattering occurs when the incident X-ray photon is deflected from its original path by an interaction with an electron. The electron gains energy and is ejected from its orbital position. The x-ray photon loses energy and continues to travel through the material in a path different than the incident one. The scattered x-ray photon has less energy (i.e. longer wavelength) than the incident photon. The energy shift depends mostly on the angle of scattering and not on the nature of the material. This is a type of incoherent scattering, because the X ray photon energy change is not always orderly and is not consistent; therefore, difficult to predict. The probability of Compton effect is directly proportional to the electron density (i.e. number of outer shell electrons), and physical density of the material, but does not depend on the atomic number, Z (unlike the photoelectric effect). Scattering may also occur by way of pair production. Pair production occurs when an electron and positron are created with the annihilation of high energy x-ray photons (greater than 1 MeV). This effect is of particular importance when high-energy photons pass through materials of a high atomic number. Other phenomena, such as Thomson scattering and Photodisintegration may be neglected for the energies of X rays typically used in radiography. Among all these phenomena happening during interaction of the X ray photons and the materials, the most notable ones happening in the medical diagnostic range of X rays, are the photoelectric effect and the Compton effect. The scattered radiation (which is mainly due to the Compton effect) is the principal source of occupational exposure to radiographers, doctors and operators, and other medical personnel. Therefore, a proper protection from both, the scatter and the primary ionizing radiation, is necessary.

X-rays along with the gamma rays are ionizing rays, which means they have the ability to remove electrons from atoms and molecules in the materials they are passing through. Thus, their ionizing activity can alter the molecules within the body and can cause harm. Frequent exposures to ionizing radiation may yield tissue damage causing DNA mutation and potentially leading to cancer and other disorders.

The best materials for radiation protection, or so-called attenuating materials of ionizing radiation, are those composed of heavy elements (high-density materials with high atomic number, Z), because of the high probability of interactions between the incident photons and the material, and consequently, the greater energy absorption from the photons by the heavy metals present in the material. Traditionally, the radiation-protective or X-ray shielding materials incorporate lead (Pb). However, Pb is associated with toxic effects on the human and animal health, as well as material disposal issues. Moreover, protection garments containing Pb tend to be uncomfortably heavy if worn for long time periods. Therefore, lead-containing non-toxic radiation-protective metals have been proposed for making radiation-attenuating materials. Some of the protective metals used for X-ray shielding, along with their atomic (Z) numbers, density (d) and K-absorption edge, are given below:

| | |
|---|---|
| (a) Tin (Sn) | Z = 50; d = 7.30 g/cm³; |
| | K-absorption edge = 29.2 keV |
| (b) Antimony (Sb) | Z = 51; d = 6.69 g/cm³; |
| | K-absorption edge = 30.5 keV |
| (c) Cesium (Cs) | Z = 55; d = 1.87 g/cm³; |
| | K-absorption edge = 36.0 keV |
| (d) Barium (Ba) | Z = 56; d = 3.50 g/cm³; |
| | K-absorption edge = 37.4 keV |
| (e) Cerium (Ce) | Z = 58; d = 6.66 g/cm³; |
| | K-absorption edge = 40.4 keV |
| (f) Gadolinium (Gd) | Z = 64; d = 7.90 g/cm³; |
| | K-absorption edge = 50.2 keV |
| (g) Tungsten (W) | Z = 74; d = 19.30 g/cm³; |
| | K-absorption edge = 69.5 keV |
| (h) Lead (Pb) | Z = 82; d = 11.36 g/cm³; |
| | K-absorption edge = 88.0 keV |
| (i) Bismuth (Bi) | Z = 83; d = 9.75 g/cm³; |
| | K-absorption edge = 90.5 keV |

Lead-containing radiation-attenuation materials are commercially-available from different providers. However, many of them still suffer from weight issues and lack of flexibility yielding to discomfort for the wearer. The lack of flexibility also results in appearance of cracks during the use, folding or storage of the protective garments, which means ineffective protection and/or short service life of such protective garments. Moreover, many of the commercial lead-containing radiation-shielding materials have narrow protection range, i.e. do not protect over a wide range of X-ray energies often used in clinical practices. Therefore, there is a need for lead-containing, flexible, crack-, abrasion- and tear-resistant radiation-protective materials, which will provide protection for the medical personnel and the patients against a broad range of X-ray energies used in medical settings.

Embodiments described herein are directed to novel light-weight metal-filled compositions and novel materials designs utilizing such compositions to provide protective garments and other protective articles with improved radiation shielding capability against primary and scatter X-rays. Certain configurations described herein include an effective amount of materials to provide radiation protection over a wide range X-ray energies, e.g., those X-ray energies which typically result when an applied tube voltage of an X-ray source is about 50 kiloVolts up to about 150 kiloVolts. While the exact level or attenuation of the X-ray radiation may vary from composition to composition, the materials desirably attenuation the X-rays to a sufficient degree to provide radiation protection to an operator and/or a patient. Geometries for measuring the attenuation (protection level) of shielding materials are described according to the IEC 61331-1:2014 standard (Protective devices against diagnostic medical x-radiation—Part 1: Determination of attenuation properties of materials). Narrow-beam geometry (NBG), broad-beam geometry (BBG), inverse-broad-beam geometry (IBG*) and modified-broad-beam geometry (BBG*) measurements can be performed according to the IEC standard. See H. Eder et al, IEC 61331-1: A new setup for testing lead containing X-ray protective clothing, Physica Medica 45 (2018) 6-11. These tests can be used to measure radiation attenuation of test samples and calculate a lead equivalence value (LEV) to compare the compositions to a lead based material. LEV values are described, for example, in Buermann. 2016 JINST 11 T09002 entitled “Determination of lead equivalent values according to IEC 61331-1:2014-Report and short guidelines for testing laboratories”; H. Eder et al, IEC 61331-1: A new setup for testing lead containing X-ray protective clothing, Physica Medica 45 (2018) 6-11; and Schopf and Pichler. Radiation Protection Clothing . . . Fortschr Röntgenstr 2016; 188: 768-775 entitled “Radiation Protection Clothing in X-Ray Diagnostics—Influence of the Different Methods of Measurement on the Lead Equivalent and the Required Mass.” LEV values are typically used to compare the measured values to a lead film of specified thickness and protection. Attenuation values are also described to compare how much radiation is attenuated by the compositions described herein.

In general, the intensity of X rays are measured without a test specimen. The test specimen is then inserted (e.g., close to the detector, far from the X ray source in BBG* mode) and the intensity of the X rays are measured. This process is repeated at different X ray tube energies (50-150 kV). These values an be used to determine attenuation ratios and, if desired, LEVs.

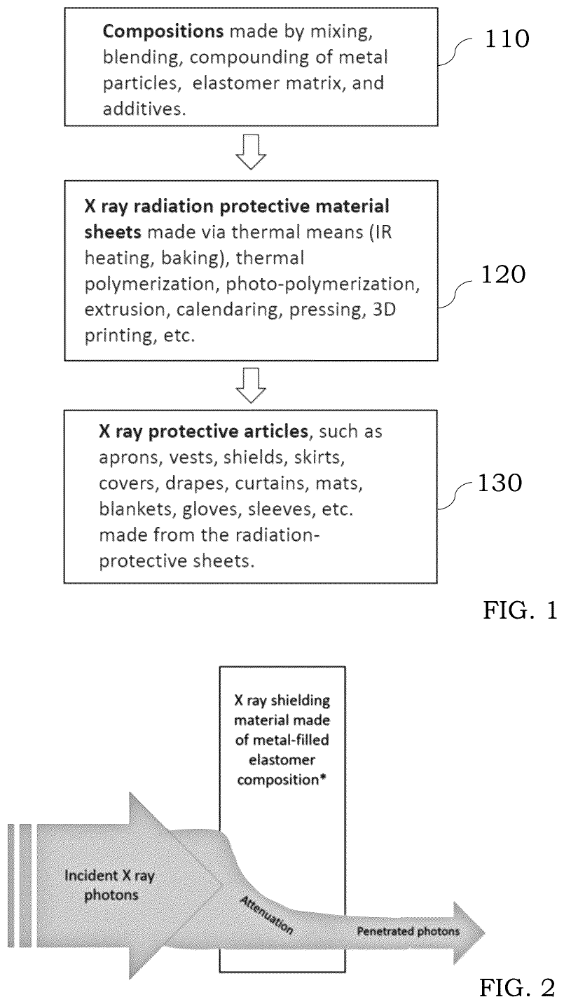

An illustrative process to produce articles that include the compositions is presented as a flowchart in FIG. 1. The various materials can be combined at step 110. Depending on the intended article configuration, the materials can be processed in various manners. For example, step 120 shows production of a sheet using various processes. The sheet can be formed into the desired article at step 1′30. The materials made of the disclosed compositions can be flexible, crack-resistant, tear-resistant, and abrasion-resistant materials and can be used to make protective garments and other protective elements with improved attenuating properties against primary and scatter X-rays. Moreover, the protective articles have tunable surface properties, such as low friction, antimicrobial and repellent properties.

Due to the ionizing nature of X-rays, the extent of penetration of X-rays into the material depends on many parameters, including the energy of incident photons, the elemental composition of the protection material, its thickness, and density among others. Regarding the elemental compositions, the type of the metals used, the metal particle size and particle size distribution, the ratio of metals used and the photon energy that the material is intended to be used are among the factors that will directly affect the effectiveness of the radiation shielding. By combining radiation protective metals with different K-edge or K absorption energies, the X-ray shielding capability can be effectively enhanced and extended over a wide range of photon energies. By increasing the number of layers in the protective material and by optimization of their compositions and thicknesses, the shielding efficacy of the protective material can be further improved. The attenuation of the incident X-rays is schematically presented in FIG. 2. Materials parameters that can effect shielding include, but are not limited to, thickness, density, metal Z number, metal particle size, metal distribution, particle size distribution, the number of layers and their interfaces, etc.

In certain configurations, a flexible matrix can be present in the compositions described herein and can be a polymeric matrix optionally including one or more plasticizers in combination with one or more lead and an X-ray radiation protective metal with Z-number greater than 50. For example, the flexible matrix can be a suspension of a polymeric matrix or other polymeric particles in a liquid plasticizer in combination with lead and a X-ray radiation protective metal with Z-number greater than 50. In some embodiments, the flexible matrix can include a plastic suspension in combination with a liquid plasticizer, lead and an X-ray radiation protective metal with Z-number greater than 50. While the exact flexible matrix materials may vary, in one illustration the flexible matrix can include a suspension of polyvinyl chloride in a liquid plasticizer, e.g., the flexible matrix can be a plastisol matrix, lead and at least one non-lead X-ray radiation protective metal with Z-number greater than 50. The non-lead protective metal can be selected from the following list of metals but not limited to: Sb, Sn, W, Bi, Ce and others. The exact amount of the particular metal(s) and its distribution can vary as noted herein.

In some embodiments, the metals are selected to be in a pure form (or substantially pure form with only minor impurities that are not easily removed), while in other embodiments the metals are in a form of a compound. For example, the metal can be present as an oxide, a carbonate, a sulfate or other type of compounds. In a particular embodiment, the metal (lead or non-lead metals) is present without other metals being present in the plastic elastomer matrix optionally with one or more additives, e.g., only lead and a single metal with a Z-number greater than 50 is present in the composition along with the materials which form the elastomer matrix. In another embodiment, a mixture of lead and at least two different heavy metals with Z-number of minimum 50 is used in the elastomer matrix for making radiation protective articles. Where mixtures are used, the mixture can include, for example, lead and any one or two or more of Sb, Sn, W, Ba, Bi₂O₃, BaSO₃, etc. For example, the mixture can include lead and one or two or more of Sb and Sn, Sb and W, Ba and Bi₂O₃, BaSO₄ and Bi₂O₃, etc. In yet, another embodiment, the protective metal(s) is an alloy or a compound of at least two metals known to be efficient for protection from X-ray and/or gamma radiation, e.g., lead can be used as a metal alloy or lead in combination with a metal alloy can be used. Examples of metal alloys include, but are not limited to, barium tungstate (BaWO₄), barium-tungsten alloy, tin tungstate (SnWO₄), bismuth tungstate (Bi₂(WO₄)₃), bismuth-tin alloy, and others. Where an alloy is present, the alloy may be present alone or combined with a metal having a Z-number greater than 50, e.g., a mixture of lead, a metal alloy and another metal with a metal having a Z-number greater than 50 can be used.

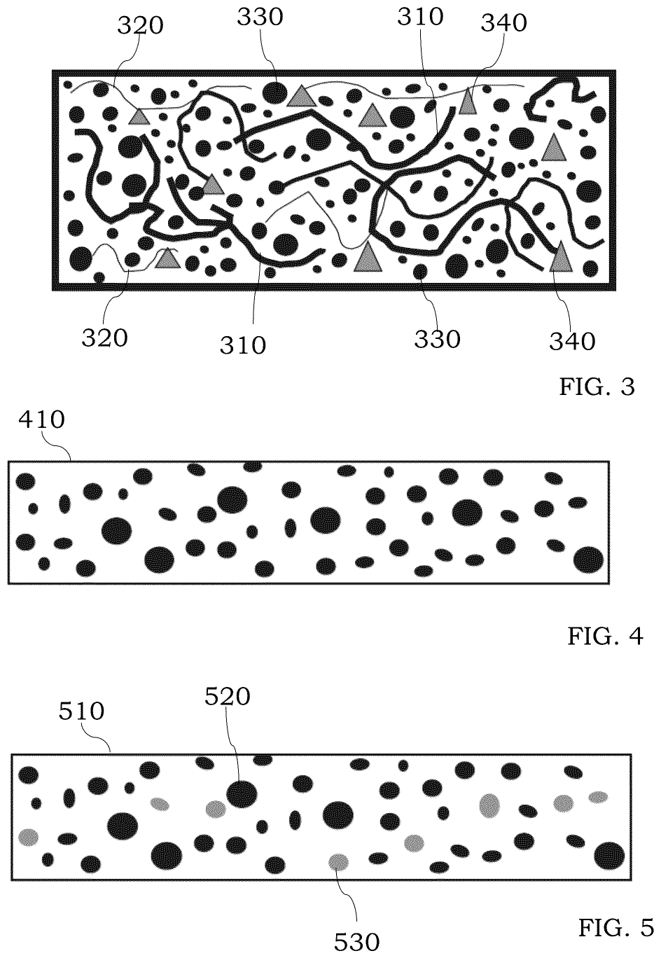

In some embodiments, the elastomeric matrix besides the metal particles comprises other components and additives, such as reinforcing fibers and functional additives, which add value to the radiation-protective material, such as impact strength, low friction, color, surface hydrophobicity, antimicrobial functionality etc. as shown in FIG. 3 For example, elastomeric polymer chains 310 (shown as thicker lines), metal shielding particles 320 (shown as circles, ellipses, etc.), reinforcing fibers 330 (shown as thinner lines) and additives 340 (shown as triangles) can be present. Illustrative additives include, but are not limited to, wetting agents, dispersants, impact modifiers, plasticizers, binders, initiators, catalysts, cross-linkers, viscosity modifiers, etc.

In a specific embodiment, the protective material, which protects from ionizing radiation, such as X-rays, gamma rays or both types of ionizing radiation, is made of one elastomeric layer comprising metal particles. For example, a single layer of material may be present. The metal particles in the layer 410 have wide particle size distribution/size to cover protection against wide range of X-rays' energies, as presented pictorially in FIG. 4.

In another embodiment, the elastomeric layer comprises at least two metal particles, e.g., lead particles in combination with one or more other particles. For example, the particles can be lead in combination with particles of Sb, Bi, W, Ba, Sn, Sb—Bi, Sb—W, Ba—Bi, Ba—W, Sb—Ba—W, Sb—Ba—Bi, Sb—Bi—W, Sb—Sn—W, etc. as presented pictorially in FIG. 5 with metal particles 510 of low atomic (Z)-number (shown in darker shading), such as Ba, Sb, etc., and metal particles 530 of high atomic (Z)-number (shown in lighter shading), such as W, Bi and others, all dispersed in the same elastomeric layer 510. The different particles need not have the same size or distribution in the layer 510.

In certain configurations, the radiation shielding composition comprises lead in combination with one of Bi, Sb, W, Sn and Ba. The Pb can be present in an amount that exceeds the amount of the Bi, Sb, W, Sn or Ba. Alternatively, the Pb can be present in an amount that is less than the amount of Bi, Sb, W, Sn or Ba. In some embodiments, the Pb can be present in the radiation shielding composition in a major amount, e.g., 50 weight percent based on a weight of the composition. In other embodiments, the ratio of Pb to the other metal(s) is 2:1, 3:1, 4:1, 5:1 or values higher than 5:1. As noted herein, the composition can include one or more polymeric materials in combination with other additives, e.g., plasticizers, viscosity modifiers, etc. The amount of the polymeric materials and other additives may vary from about 10 wt % to about 50 wt %. The metal:polymer ratio can vary from 50:50 to 95:5, and more preferably from 70:30 to 90:10 depending on the metal particles' size, shape and particle size distribution. The metal volume percentage in the compositions vary from 20 to 40% by vol, and more preferable from 30 to 40% by vol. Additives, such as initiators, catalysts, processing aids, rheological modifies, plasticizers, lubricants, cross-linkers, curing agents, chain extenders, colorants, heat- and UV-stabilizers, slip agents, wetting agents, dispersants, antioxidants, compatibilizers, adhesion promoters, and others can be used in compositions comprising Pb and one of Bi, Sb, W, Sn and Ba.

In other configurations, the radiation shielding composition comprises lead in combination with two of Bi, Sb, W, Sn and Ba. The Pb can be present in an amount that exceeds the total amount of the Bi, Sb, W, Sn or Ba. Alternatively, the Pb can be present in an amount that is less than the total amount of Bi, Sb, W, Sn or Ba. In some embodiments, the Pb can be present in the radiation shielding composition in a major amount, e.g., 50 weight percent based on a weight of the composition. In other embodiments, the ratio of Pb to the other metal(s) is 2:1, 3:1, 4:1, 5:1 or values higher than 5:1. As noted herein, the composition can include one or more polymeric materials in combination with other additives, e.g., plasticizers, viscosity modifiers, etc. The amount of the polymeric materials and other additives may vary from about 10 wt % to about 50 wt %. The metal:polymer ratio can vary from 50:50 to 95:5, and more preferably from 70:30 to 90:10 depending on the metal particles' size, shape and particle size distribution. The metal volume percentage in the compositions vary from 20 to 40% by vol, and more preferable from 30 to 40% by vol. Additives, such as initiators, catalysts, processing aids, rheological modifies, plasticizers, lubricants, cross-linkers, curing agents, chain extenders, colorants, heat- and UV-stabilizers, slip agents, wetting agents, dispersants, antioxidants, compatibilizers, adhesion promoters, and others can be used in compositions comprising Pb and two of Bi, Sb, W, Sn and Ba.

In certain embodiments, the radiation shielding composition comprises lead in combination with three or more of Bi, Sb, W, Sn and Ba. The Pb can be present in an amount that exceeds the total amount of the Bi, Sb, W, Sn or Ba. Alternatively, the Pb can be present in an amount that is less than the total amount of Bi, Sb, W, Sn or Ba. In some embodiments, the Pb can be present in the radiation shielding composition in a major amount, e.g., 50 weight percent based on a weight of the composition. In other embodiments, the ratio of Pb to the other metal(s) is 2:1, 3:1, 4:1, 5:1 or values higher than 5:1. As noted herein, the composition can include one or more polymeric materials in combination with other additives, e.g., plasticizers, viscosity modifiers, etc. The amount of the polymeric materials and other additives may vary from about 10 wt % to about 50 wt %. The metal:polymer ratio can vary from 50:50 to 95:5, and more preferably from 70:30 to 90:10 depending on the metal particles' size, shape and particle size distribution. The metal volume percentage in the compositions vary from 20 to 40% by vol, and more preferable from 30 to 40% by vol. Additives, such as initiators, catalysts, processing aids, rheological modifies, plasticizers, lubricants, cross-linkers, curing agents, chain extenders, colorants, heat- and UV-stabilizers, slip agents, wetting agents, dispersants, antioxidants, compatibilizers, adhesion promoters, and others can be used in compositions comprising Pb and three or more of Bi, Sb, W, Sn and Ba.

In another embodiment, the elastomer is a polyolefin flexible matrix with at least one non-lead radiation-protective metal. In some embodiments, the polyolefin is composed only of one type of monomer units, such as but not limited to ethylene, propylene, butene, etc. In other embodiments, the polyolefin elastomer is made of a copolymer comprising at least two different types of repeating monomer units, such as ethylene and vinyl, or others. In general, elastomers are polymers exhibiting viscosity and elasticity at the same time; therefore they are also known as viscoelasticity materials. The molecules of elastomers are held together by weak intermolecular forces and generally exhibit low Young's modulus and high yield strength or high failure strain in addition to high strain (elongation). Elastomers are natural or synthetic polymers having elastic properties. They can deform elastically under tensile and compressive stress, but when the stress is removed, they return to their original, undeformed shape. This is due to the weak intermolecular forces between the molecules of elastomers, and elastomers generally exhibit low Young's modulus and high yield strength or high failure strain. In particular embodiments, one or more types of elastomers are used for preparation of radiation-shielding compositions. As example only, the elastomer can be selected from the following group, but not limited to natural rubber, polyurethane, polybutadiene, silicone, neoprene, polyolefin elastomer, etc. or their combination. Particular examples include but are not limited to EPM (ethylene propylene rubber, a copolymer of ethene and propene), EPDM rubber (ethylene propylene diene rubber, a terpolymer of ethylene, propylene and a diene-component), epichlorohydrin rubber, polyacrylic rubber, fluorosilicone rubber (FVMQ), ethylene-vinyl acetate (EVA), plastisol, etc. In particular embodiments, the matrix is a thermoplastic elastomer (TPE). The flexible macromolecular chains form mostly amorphous regions, but can exhibit also crystalline microdomains by local ordering of the chains or by physical bonds (leading to physical cross-linking).

In particular embodiments, thermoplastic elastomers (TPE) are used as the matrix where the metal particles are dispersed. TPEs combine the processing advantages of thermoplastics with the performance properties of elastomers. All TPEs are composed of crystalline and amorphous domains. These can be physical blends or alloys of crystalline and amorphous polymers, or they can be block copolymers, which are chemical mixtures of blocks of crystalline and amorphous domains in the polymer chain. The hard blocks are responsible for the plastic properties of the final product in the case of TPEs, including easy processing and temperature resistance and material properties such as tear and tensile strength or chemical resistance. Adhesion is also determined by these properties. The soft blocks are responsible for the elastomeric or elastic properties. They determine material properties such as toughness and flexibility as well as the extent of deformation (elongation, strain). TPE shows rubber elasticity at room temperature and can be plasticized at high temperature. Therefore, this type of polymer material combines the characteristics of thermoplastic rubber and thermoplastic plastics. The basic structural feature of a TPE polymer chain is that it is comprised of some plastic segments (hard segments) and rubber segments (soft segments) originating from different chemical compositions. The interaction between the hard segments is sufficient to condense into microdomains (glassy or crystalline microdomains) to form physical crosslinks between molecules While the soft segment is a segment with high rotation ability and flexibility.

In certain embodiments, polyolefinic TPE are used as matrix. These materials are defined as compounds (or mixtures) of various polyolefin polymers, often mixture of semicrystalline thermoplastics and amorphous elastomers. Some polyolefin TPEs are composed of polypropylene (PP) and a copolymer of ethylene and propylene called ethylene-propylene rubber. A common rubber of this type is called ethylene propylene diene monomer rubber (EPDM), which has a small amount of a third monomer, a diene which provides a small amount of unsaturation in the polymer chain that can be used for cross-linking. In other embodiments, the polyolefin TPE is a copolymer of ethylene and octene, the later providing excellent flexibility and elasticity of the material.

In some embodiments the TPE material can be polyolefin (PO), diene, polystyrene (PS), polyvinyl chloride (PVC, plastisol), polyurethane (PU), polyester (PET), silicone, etc. or their combination in a form of blend, alloy, copolymer, inter-penetrating network (IPN), semi-inter-penetrating network (SIPN), inter-connected network (ICN), etc.

Polyurethane TPE (PU) is a thermoplastic polyurethane rubber composed of urethane-bonded hard segments and polyester or polyether soft segments with excellent mechanical strength, chemical resistance and flexibility.

In some embodiments, the matrix is a silicone-based polymer. Silicone polymers (polysiloxanes) contain repeated Si—O bonds in the molecular structure. Polysiloxanes, such as silicone oil, silicone rubber and/or silicone resin have great properties, viz. weather resistance, temperature resistance, aging resistance, hydrophobicity, etc. Furthermore, in other embodiments, the matrix is a silicone-modified matrix. The hard fraction of the matrix can be any thermoplastic or thermosetting polymer, while the soft fraction is polysiloxane. The hard and the soft fractions of the matrix can be incorporated in a single polymer, such as block or graft copolymer of the following types, but not limited to AB, ABA, (ABBA)n, (AB)n, and so on, or their combination, where A is the hard segment, while B is the soft elastomer segment.

In other embodiments, the hard and the soft fraction of the matrix can be similar or different chemical species. e.g. a polyolefin polymer and a polysiloxane. In a particular embodiment, polydimethylsiloxane (PDMS) is the soft segment component of the matrix.

In other embodiments, the matrix material is a thermosetting elastomer (TSE), where different degrees of chemical cross-linking are present. These elastomers exhibit lower strain (elongation), but usually are more stable to external forces.

In certain embodiments, unsaturated rubbers are used for preparation of the composition, which subsequently is cured by sulfur vulcanization or by other means known in the art.

In other embodiments, thermosetting elastomers (TSEs) are used as matrix for the dispersed metal particles. These materials provide excellent strength and less stretching when deformed, which is great for particular radiation shielding applications, such as curtains, etc.

In some embodiments, the elastomeric matrix is a natural rubber.

In other embodiments, the matrix is a dynamic vulcanized thermoplastic elastomer. It is prepared by dynamic vulcanization of a mixture of a thermoplastic polymer and elastomer. By means of the vulcanization process of a vulcanizing agent (crosslinking agent) under strong mechanical shear stress, the crosslinked rubber particles are dispersed in a continuous thermoplastic matrix and finely dispersed particles. Compared with ordinary thermoplastic elastomers, the rubber components are completely vulcanized and uniformly dispersed in the thermoplastic matrix, so that they have good physical and mechanical properties, processing properties, chemical and heat resistance.

In certain embodiments, phase-change-materials (PCMs) are added to the radiation-shielding composition. PCMs are able to absorb, store and release large amounts of latent heat over a defined temperature range when the material changes its phase or state. The heat absorption by PCMs results in a delay in microclimate temperature and thus, can be used to provide district cooling to the wearer of the garment containing PCMs. This will result in preventing over-heating of the wearer and improved wearing comfort.

In certain embodiments, the non-Newtonian character of the elastomer or the final composition (elastomer, additives, metals) is used to make the final material structure or design, i.e. metal “ordering” in sublayers. As example only, two different types of granules can be used, granules X and Y, which have very distinct pseudoplastic (shear-thinning) behaviors. In another example, the granules X and Y differ in the type of their rheological behavior, e.g. granules A have pseudoplastic and the other granules (granules B) have dilatant (shear-thickening) behavior. In yet other examples, the granules X and Y can exhibit pseudoplastic and Bingham plastic behavior, or thixotropic (time-dependent shear-thinning) and rheopectic (time-dependent shear-thickening) behavior, or any other combination of different rheological behaviors. The very distinct rheological behaviors of granules X and Y result in different structure designs in the final materials made by e.g. sheet extrusion. In some instances, the final extruded sheets have co-continuous two-phase system structures, sub-layer ordered structure, etc.

In some embodiments, the matrix material is an elastomer comprising at least two polymers with different viscoelastic properties.

Additional post-processing steps are disclosed to add protective or other functionality to the material or garment, such as superhydrophobic, self-cleaning, self-healing, cooling or other functionality.

In particular embodiments, elastomers with shape memory effects are able to deform and recover their shapes moving between original and temporary states. This type of shape-memory elastomers (SMEs) are a class of intelligent materials characterized by their ability to deform and recover shapes under applied force and/or other external stimuli. Heat and ultraviolet radiation are the most common external stimuli. elastomers with shape memory effects are able to deform and recover their shapes moving between original and temporary states.

In some embodiments, the matrix is made with low viscosity polymer, metal particles and all additives, of which at least one of the ingredients is a shear-thickening material, i.e. a non-Newtonian fluid, which show an increase in viscosity with increasing stress or shear rate. Such shear-thickening material can be prepared by dispersing concentrated colloidal suspensions of solid particles in a liquid, where the transition from low to very high viscosity is based on forming transient aggregates upon shear. As an example only, dispersing colloidal silica particles (micron or sub-micron size particles) dispersed in an oligomer or polymer with low molecular weight (e.g. oligomer of about 200 Daltons) can be used.

In some embodiments, the expanded graphite or nanoclay—materials with high specific surface area and porosity are used. The porous material is doped, impregnated, or filled with radiation protective metal particles by any method or process known in the art. Such doped, impregnated, or filled porous material is then laminated, co-extruded, or added to an elastomer to make the final radiation protective sheet.

In some embodiments, the flexible matrix can be a 2D planar or 3D networks. It is known in the art that certain metals, such as Bi, W, Sb and others can form 2D (planar) ordering (network) and have found applications mainly in optics, electronics, semiconductor or photonic industry. In certain aspects of the invention, such 2D metal networks are utilized to prepare thin light-weight X ray shielding materials. Due to the planar ordered metal network, the metals are uniformly ordered across the plane, thus, no need of thick materials. Such 2D metal networks can be embedded between 2 elastomeric layers, made of e.g. plastisol, PDMS, TPO, etc. The 2D-metal networks can be produced by any method known in the art, such as but not limited to molecular beam epitaxy, pulsed laser deposition, electron-beam (e-beam) evaporation, magnetron sputtering, thermal evaporation, flash vaporization and other physical vapor depositions, chemical vapor deposition, wet chemical processes, exfoliation, etc. 2D bismuth, 2D tungsten, antimonene, transition metal dichalcogenides (TMDCs), e.g. tungsten dichalcogenide and other metals.

In some embodiments, at least one of the X ray protective metals, is added as metal-organic framework (MOF) to the composition, such as bismuth MOFs, tungsten MOFs, lead MOFS, etc.

In particular embodiments, the protective metal is added in a form of a metal complex, or the metal complex is formed during mixing the metal with the polymers and additives under certain mixing conditions (temperature, pressure, shear rate).

In yet another embodiment, the metals and/or other constituents of the composition are in a form of foam, micro-foam and/or other 3D structure (with order or no particular order).

In another embodiment, the flexible matrix is a urethane elastomer.

In another embodiment, the flexible matrix is a natural rubber or other materials comprising one or more isoprene units.

In other embodiments, the matrix is selected from one or more materials in the following list of elastomers: neoprene, nitrile butadiene rubber, silicone, nitrile butadiene, styrene butadiene, polyester urethane, polyether urethane, acrylate urethane, fluoro-silicone, etc.

In some configurations, combinations of different elastomers can be used to make the radiation-protective material.

In certain embodiments, hydrogels, such as polyvinyl alcohol (PVA), ethylene vinyl acetate (EVA), ethylene propylene diene monomer (EPDM), and others are used as flexible matrix where the protective metals are dispersed. In yet other embodiments, epoxy resins, polyesters, polyamides, co-polyamides and other materials are used as a matrix material alone, or in a combination with an elastomer, or as a coating of the elastomeric layer.

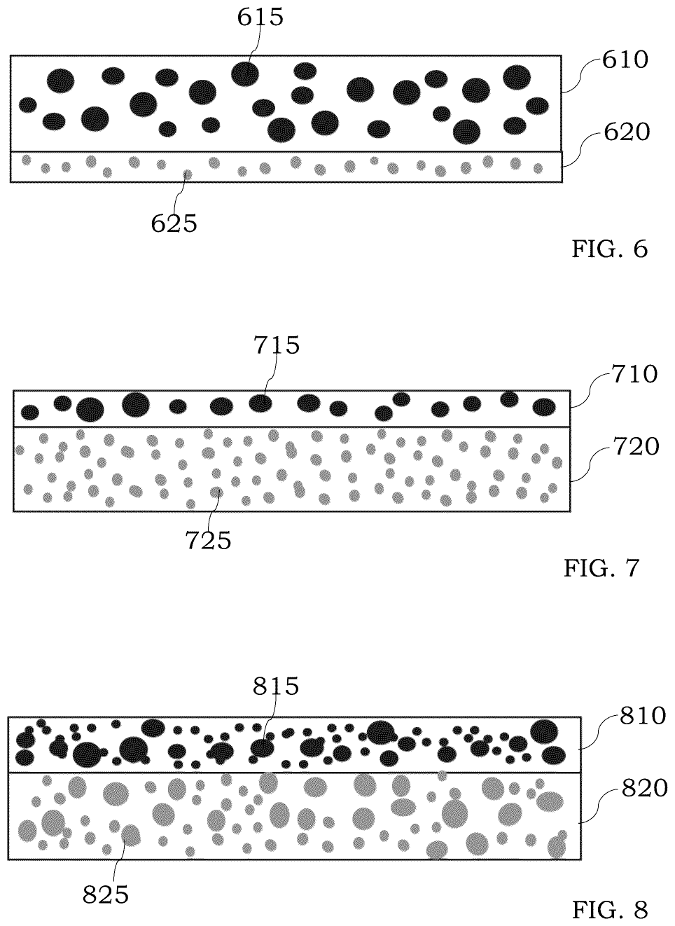

In some embodiments, the radiation-protective material is made of at least two separate layers. In certain embodiments, a layer can be separated from another layer by an interface between the two layers. The layers may be bonded to each other or may be physically separated from each other. In some configurations, a protective material may comprise two separate layers, which can be made by e.g. co-extrusion, or casted one on top of other, and which are bonded by chemical and/or physical bonds. In other instances, physically-separated layers, not bound by any bonds, can be placed on top of each other in the article to provide enhanced protection. Moreover, the two-physically-separated layers can be sewed together, laminated to each other or can be held together by application of an adhesive, laser welding, melting or other materials. For example, each layer comprises only one type of metal protective particles, as presented in FIGS. 6, 7 and 8. An example only, the material comprises a thicker layer 610 with low atomic (Z) number metal particles 615, such as Sb or Ba, and a thinner layer 620 with high atomic (Z) number metal particles 625, such as W or Bi (FIG. 6). One or both of the layers 610, 620 can include lead as noted herein. The particles of both metals can be of similar particle size distributions or with particles with different particle size distributions. Low Z-metals here are defined as those having Z-number of 60 or below, medium Z-metals are defined here as those having Z-number of 70 or below, but greater than 60, while the high Z-metals are the metals having Z-number greater than 70. As example only, the low Z-metal-consisting layer comprises particles in e.g. 10-100 μm average particle diameter range, while the other layer with high Z-metal particles comprises particles with average particle sizes of less than 5 μm. Another example is one layer, such as the low-Z-metal-filled layer 710 comprises micrometer-size particles 715 and is thinner, while the second layer 720 filled with high Z-metal particles 725 is thicker and comprises particles of sub-micrometer sizes (FIG. 7). One or both of the layers 710, 720 can include lead as noted herein. Such layers are especially useful when a wide range radiation protection is needed, so one of the layers is optimized for the higher energy range, while the second layer, usually with metal of higher atomic (Z) number, is optimized for the lower energy rage, where scatter (secondary, tertiary radiation) radiation occurs. In yet another embodiment, both layers, one layer 810 comprising the low-Z-metal materials 815 and the other layer 820 comprising high-Z-metal materials 825 have wide particle size distributions as shown in FIG. 8. One or both of the layers 810, 820 can include lead as noted herein.

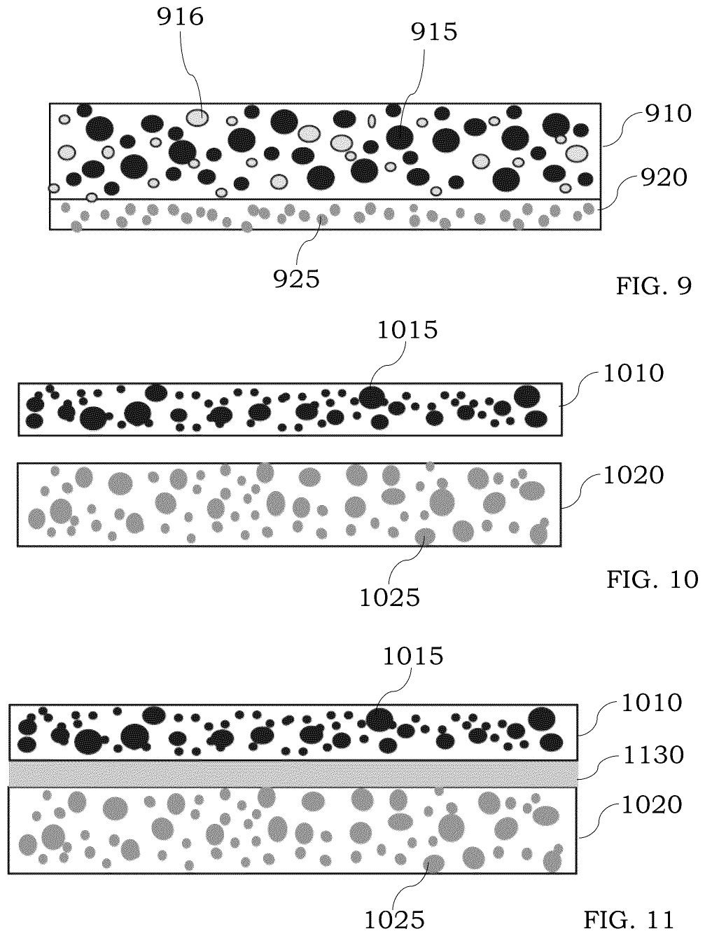

In some embodiments, the radiation protective material comprises two separate layers, and at least one of the layers comprises at least two different protective metals. By way of example only, one layer 910 comprises low-Z particles 915 (darker shading) and medium-Z metal particles 916 (lighter shading), such as Sb and particles, and the second layer 920 comprises high-Z-metal particles 925, such as W particles (see FIG. 9). One or both of the layers 910, 920 can include lead as noted herein.

In certain embodiments, the radiation protective material comprises more than two layers in order to provide protection over a broad range of X-ray energies, e.g. 10-150 kV. Besides the increased number of layers and the interfaces present, the metal particle sizes are further optimized to yield improved shielding efficacy. For instance, the layer in the material sheet which will be close to the X-ray source can be filled with (or include) the lower Z-metals, and the layers in the sheet which will be closer to the body can be filled with (or include) higher Z-metals. Also, going from the outer sheet layer (close to the X-ray source) towards the sheet layer close to the body, the particles can decrease in size.

In some embodiments, the radiation protective material comprises at least two separate layers which are chemically bonded to each other, while in other embodiments, the two separate layers are bound by physical forces, stitching or otherwise not bound to each other in a covalent manner. In yet, other embodiments, the two separate layers are not bound at all, but represent two distinct layers, which are put together, on top of each other, at a later stage when the radiation protective sheet is made, as presented in FIG. 10. A layer 1010 with low atomic Z-number particles 1015 is shown as being separate from a layer 1020 with high atomic Z-number particles 1025. Furthermore, by example only, to hold these two distinct layers together, they can be sewed together on the edges or anywhere else, or can be hold together by a thin adhesive layer 1130, as presented in FIG. 11. One or both of the layers 1010, 1020 can include lead as noted herein.

In a particular embodiment, the elastomeric material is a self-healing elastomer. As example only, the self-healing elastomer can be a functionalized polydimethylsiloxane (PDMS), which due to its dynamic hydrogen bonds and dynamic disulfide bonds exhibit self-healing properties. The articles made of a radiation-protective material comprising self-healing elastomer filled with metal particles have the ability to self-repair after cracks, punches or other defects generated in the radiation-protective article, such as apron, vest, skirt, thyroid shield, and so on, during normal wear or storage. Another example of self-healing elastomer includes polyvinyl alcohol (PVA) hydrogel prepared by a freezing/thawing method. Such hydrogel article has the ability to self-repair at ambient temperatures without the need of healing agent or any other external stimulus after it has been torn, cracked or being cut during normal wear. In yet another example, the self-healing elastomer, besides the metal fillers, comprises capsules of an additive (catalyst, initiator) that promotes repair of the elastomeric matrix after experiencing a crack, cut, or other type of defect. In some embodiments, the self-healing elastomer is able to repair without external stimuli, while in other embodiments, the self-healing elastomer is capable to self-repair after triggering by an external stimulus, such as heat, light, pH, etc. In other embodiments, vitrimers, also known as materials with adaptable or dynamic covalent networks are used in the radiation protective materials and compositions described herein. Vitrimers are capable of self-healing and/or shape-reprocessing, when triggered by certain factors, such as elevated temperature, certain pH, light, moisture, etc., when dynamic covalent bond exchange reactions occur.

In a particular embodiment, the elastomer matrix is a metal-filled shape-memory elastomer. The article made of such elastomer, e.g. a radiation-shielding vest or a radiation-shielding apron, when is being triggered by a heat (body heat), or light, or both, will adjust to the body shape of the wearer.

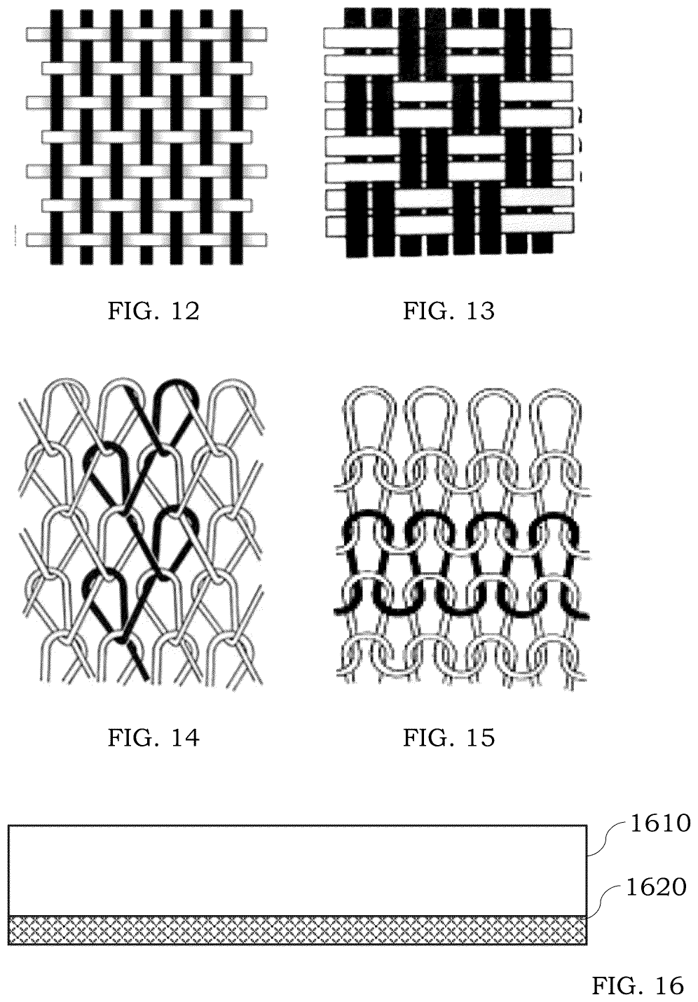

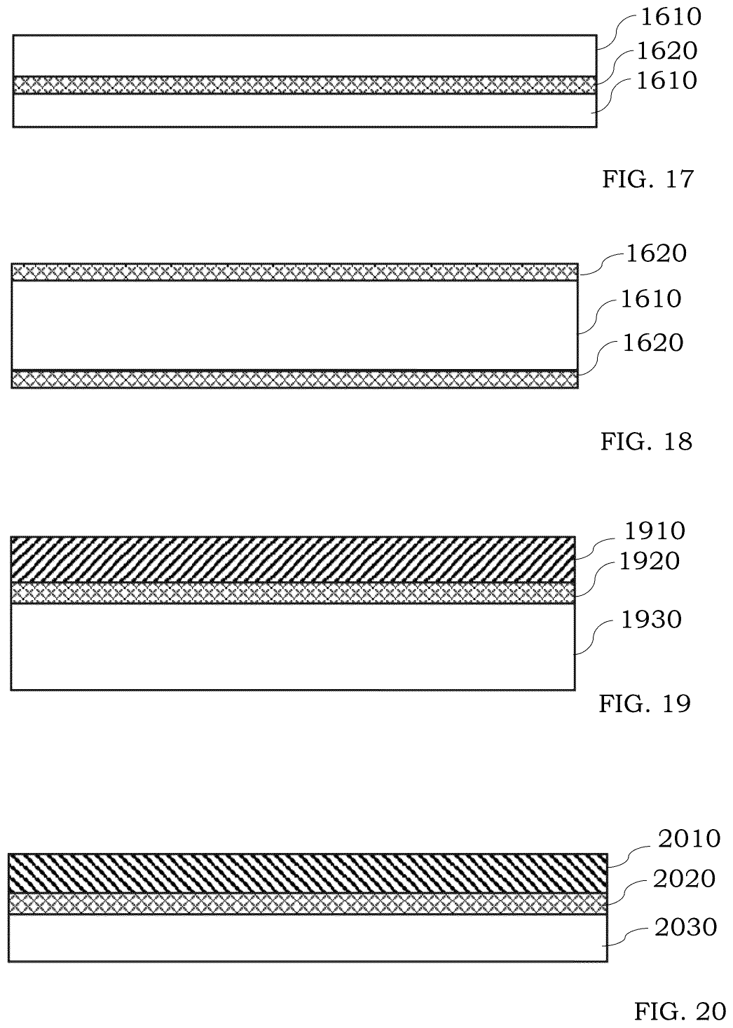

In certain embodiments, the protective material sheet comprises a hybrid structure, i.e. a structure comprising layers made of at least two different material classes where at least one of the materials of the hybrid structure can include one or more of the metal compositions described herein. For instance, the radiation-protective material sheet comprises a fabric layer, which can be a non-woven, a woven (FIGS. 12 and 13) or a knitted fabric (FIGS. 14 and 15). By means of example, a hybrid structure can be selected from the following but is not limited to a hybrid structure comprising an elastomer 1610 and a fabric 1620 (FIGS. 16, 17 and 18), a thermoplastic elastomer 1930, a fabric 1920 and a thermoset elastomer 1910 (FIG. 19), an elastomer 2020, a fabric 2020 and a hydrogel 2010 (FIG. 20), etc. Other combinations and arrangements of these materials are possible. Any one or more of the layers shown in FIGS. 16-20 can include lead as desired.

In a particular aspect, the radiation shielding material comprises a fabric. In some embodiments, the fabric serves as a tear-and crack-resistant layer. In a particular embodiment, the fabric is embedded (impregnated) in the elastomer layer comprising the protective metal particles. In another embodiment, the elastomer layer is cast on a fabric carrier layer. In yet another embodiment, the elastomer is sandwiched between two fabrics. In some embodiments, the fabric separates two elastomer layers, which can be the same or can be different. For example, the elastomer layers can be made by different means, such as but not limited to casting, dip-coating, spraying, etc.

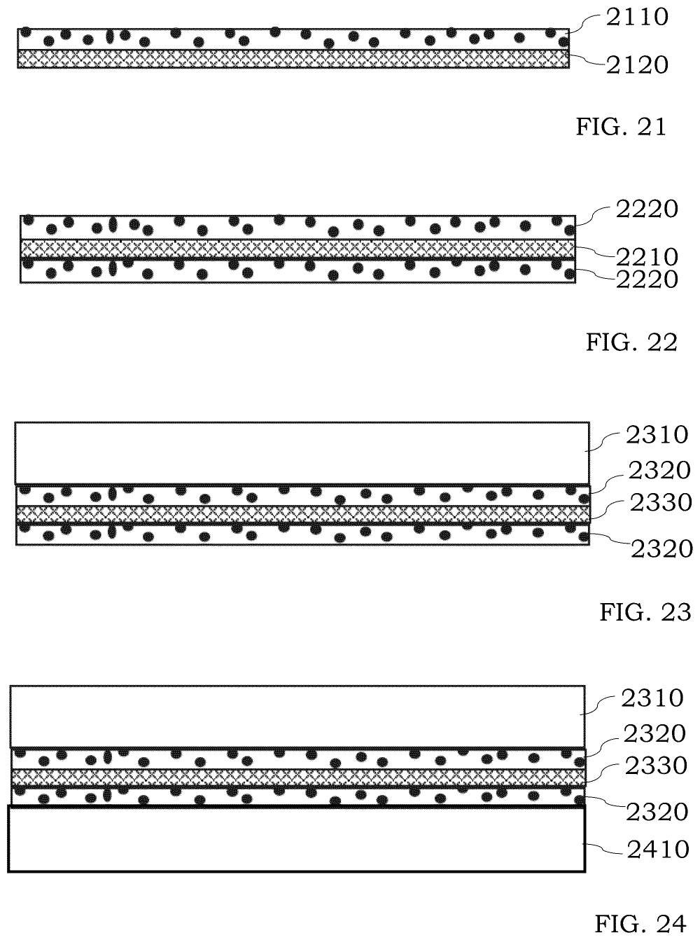

In yet other embodiments, the fabric layer can serve as a carrier for the protective metal particles. As example only, a fabric layer is coated with a coating containing the protective metal particles, as presented in FIGS. 21 and 22. In a particular embodiment, the fabric coating 2120 contains one type of metal particles (FIG. 21) on a fabric layer 2110, while in other embodiments it comprises at least two different types of radiation-protective metal particles, e.g. low-Z and high Z-metal particles. One or both of the layers 2110, 2120 can include lead. In yet another embodiment, a fabric 2210 is coated with a metal-particle-containing coating 2220 on both sides (FIG. 22), by a method known in the art, such as but not limited to dip-coating, electrostatic deposition (precipitation), spraying, etc. The metals on both sides of the fabric 2210 can be selected to be the same type or of different types of metals, e.g. one of low atomic (Z) number, and the other of high atomic (Z) number. One, two or all three of the layers 2210, 2220 and 2230 can include lead.

In some embodiments, the fabric itself is used to make the radiation-protective articles, such as personal garments, vests, shields, skirts, aprons and so on, for protection against X-ray radiation. In other embodiments, the metal-coated fabric is added to an elastomeric layer 2310, and as such is used to make garments and other articles for protection from X-rays (FIG. 23). For example, an elastomeric layer 2310 can be present a metal filled layer 2320 that sandwiches a fabric layer 2330. The elastomeric layer can also contain the protective metal particles, same type or a different type metal than those on the fabric layer. Any one or more of the layers 2310, 2320, 2330 can include lead. In yet, another embodiment, the metal-coated fabric is used in addition to an elastomer layer and/or a hydrogel layer 2410, such as PVA, which layers comprise same metal particles or different metal particles than the metal coated onto the fabric (FIG. 24). If desired, the hydrogel layer 2410 can include lead particles.

In particular embodiments, the fibers and/or the threads used to make the fabric can contain the metal particles. The metal-containing fibers are made from a polymer, such as polyethylene (PE), polypropylene (PP), polyester terephthalate (PET), polyethylene naphthalate (PEN), nylon, polyacrylonitrile (PAN), polyamide, polycarbonate (PC), aramid, combinations thereof and others, containing the particles, or the fibers are being sprayed or coated with the metal particles after being produced. Knitted, woven and non-woven fabrics are then made of such fibers, which fabrics are used with or without elastomer layer to produce radiation-protective garments.

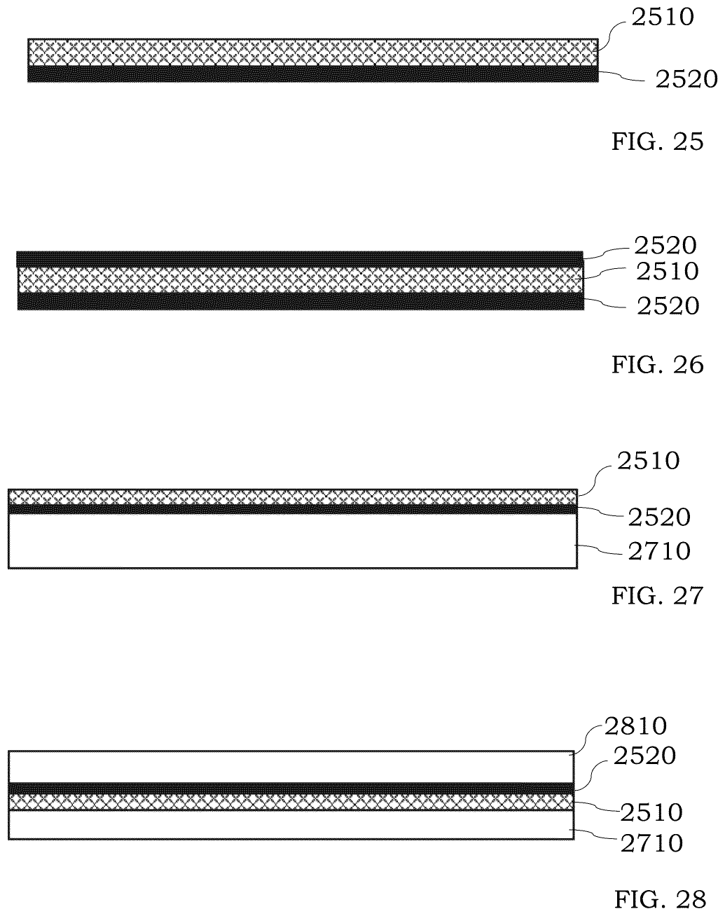

In some embodiments, the fabric is coated with a continuous metal coating, where the metal is selected from the radiation-protective metals, such as Pb, Sb, Ba, Ce, W, Bi, and others. In some embodiments, the metal coating applied to the fabric includes leads in combination with at least one of Sb, Ba, Ce, W, Bi, and other metals. The fabric 2510 is coated with a thin metal layer 2520 on one side or both sides (FIG. 25 and FIG. 26) by any deposition method known in the art, such as but not limited to thermal spray, vacuum deposition methods (CVD, PVD, magnetron sputtering, thermal evaporation, e-beam evaporation), plasma-assisted methods, ALD, LbL, electro-static deposition, etc. In other embodiments, elastomer and/or hydrogel layers (2710, 2810) can be added to the metal-coated fabrics as presented in FIG. 27 and FIG. 28. Layers 2710, 2810 can independently be an elastomer or a hydrogel.

In another embodiment, the radiation protective sheet comprises at least two layers, and one is a hydrogel layer, such as PVA filled with at least one type of protective metal particles, such as Pb, Bi, Ba, W, Ga, Sb, Ce, etc., which metals can be in their pure forms or in a form of a compound. If desired, the PVA can be filled with lead particles in combination with one or more of Bi, Ba, W, Ga, Sb, Ce, etc. PVA is a water-soluble material and the articles made of a hydrogel layer can be washed in water, where the PVA layer dissolves and the metal particles can be collected and used again.

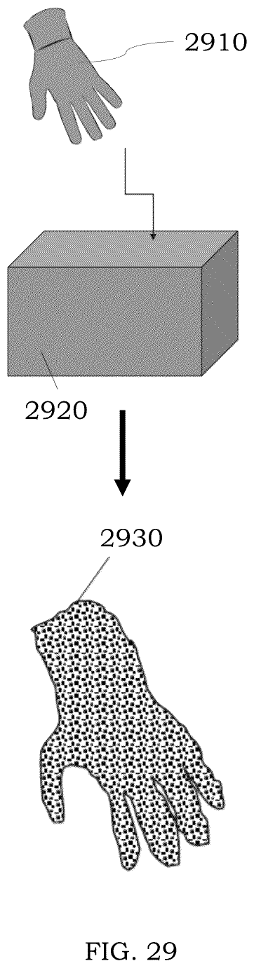

An illustrative process is shown schematically in FIG. 29 with a fabric or rubbery glove 2910 to be made into radiation-protective gloves. The glove 2910 is immersed in PVA water solution with dispersed radiation-protective metal particles in a tank 2920, and then dried with e.g. heat gun, hand drier or other means and are ready to be used as a protective glove 2930. After such coated radiation protective gloves have been used by the operator or a doctor, they can be disposed in a water tank, where the PVA layer dissolves and the metal particles are recovered and can be used again.

In some embodiments, the elastomer matrix can be prepared by partially using grinded already used and disposed radiation-protective materials. The recycled content can be in the range between 10 and 80% wt, and preferably in the range between 30 and 50% wt. The recycled content can include a similar or different metal as a virgin radiation shielding composition.

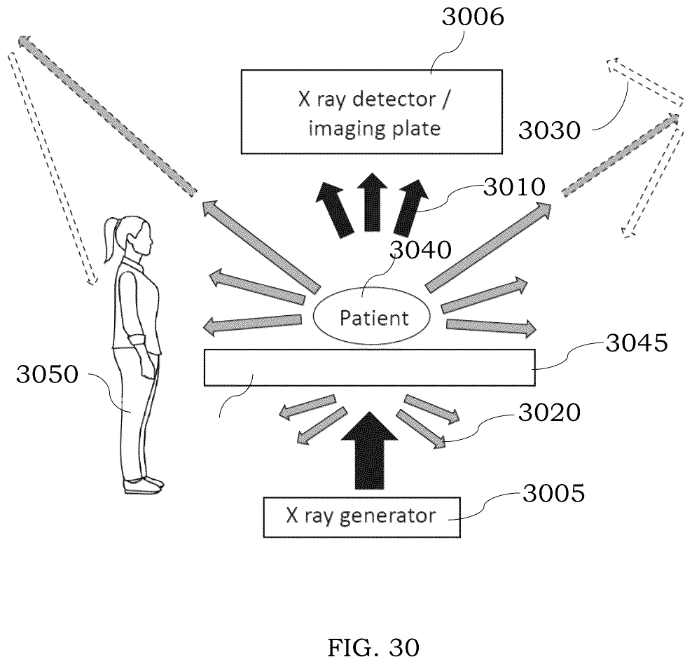

The novel radiation-protective materials described herein and the articles made of such materials provide protection for patients, operators, nurses and doctors in X-ray diagnostic, imaging and interventional clinical settings against primary and scattered X-rays, ranging from about 10 kV to about 140 kV. A schematic presentation of primary X-rays 3010, secondary X-rays 3020 and tertiary X-rays 3030 generated in a medical imaging setting is shown in FIG. 30. An operator 3050 is shown for reference. The operator 3050 typically uses the X-rays in some procedure performed on a patient 3040 on a table 3045. X-rays are provided from an X-ray generator 3005 and detected an X-ray detector 3006. The operator 3050 is typically exposed to the scattered X-rays (secondary X-rays 3020 and tertiary X-rays 3030), and the patient 3040 may be exposed to all three types of X-rays.

In particular embodiments, the protective garments and other protective elements can be produced using the single-, bi- and multi-layer protective sheets, which sheets are previously prepared using the disclosed metal-filled elastomeric compositions. Variety of protective garments and other articles, such as but not limited to barriers, pads, drapes, thyroid collars, gonad shields, breast shields, shin guards, accessories, protective caps, hoods, sleeves, gauntlet sleeves, vests, hands protection, gloves, leg wraps, aprons, skirts and others are made of the protective sheet materials. In some embodiments, the radiation-protective sheets are covered with different fabric materials which enable comfort for the wearers. Various designs and design elements are also included to enable breathability of the garment, soft touch, etc. In other embodiments, the X-ray protective garments made with e.g. aramid fibers are designed to protect the body from X-ray energies, but also provide bullet-proof protection of military personnel.

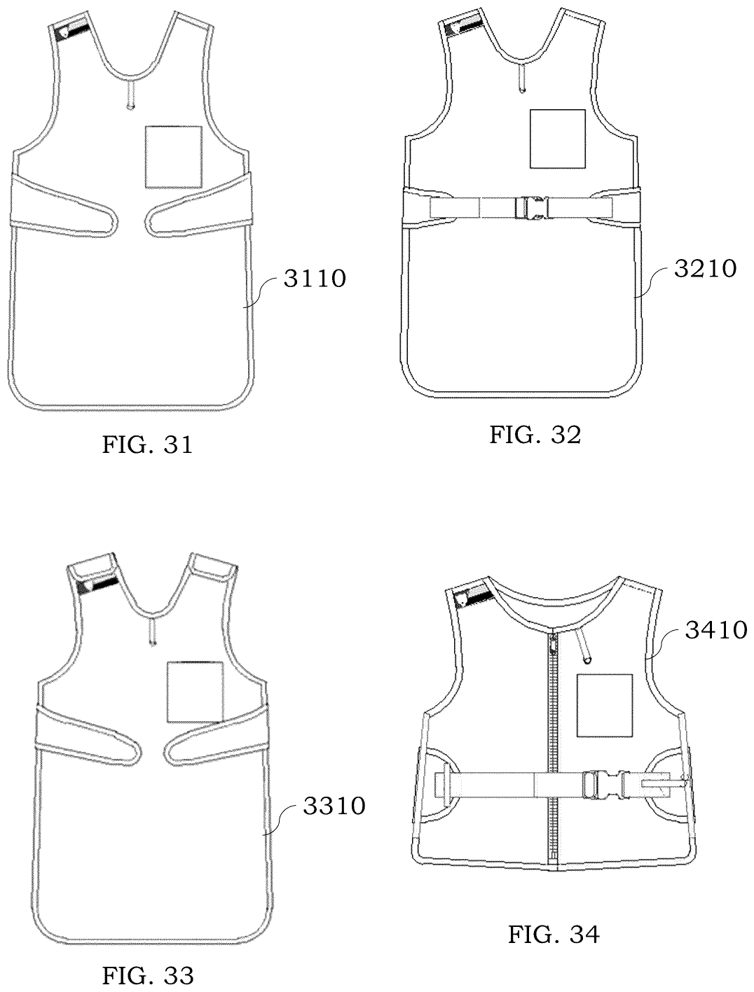

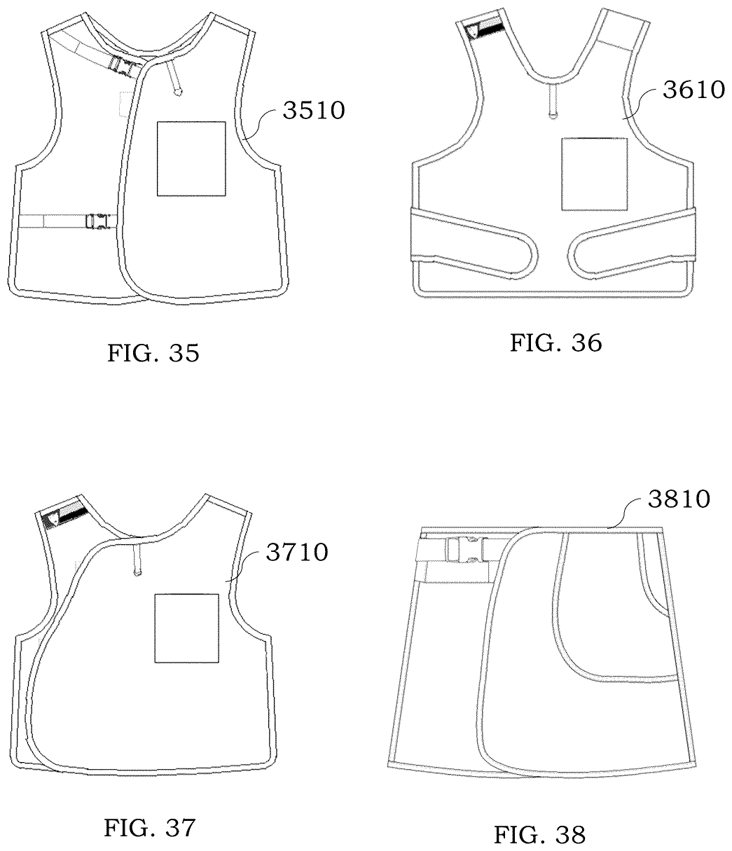

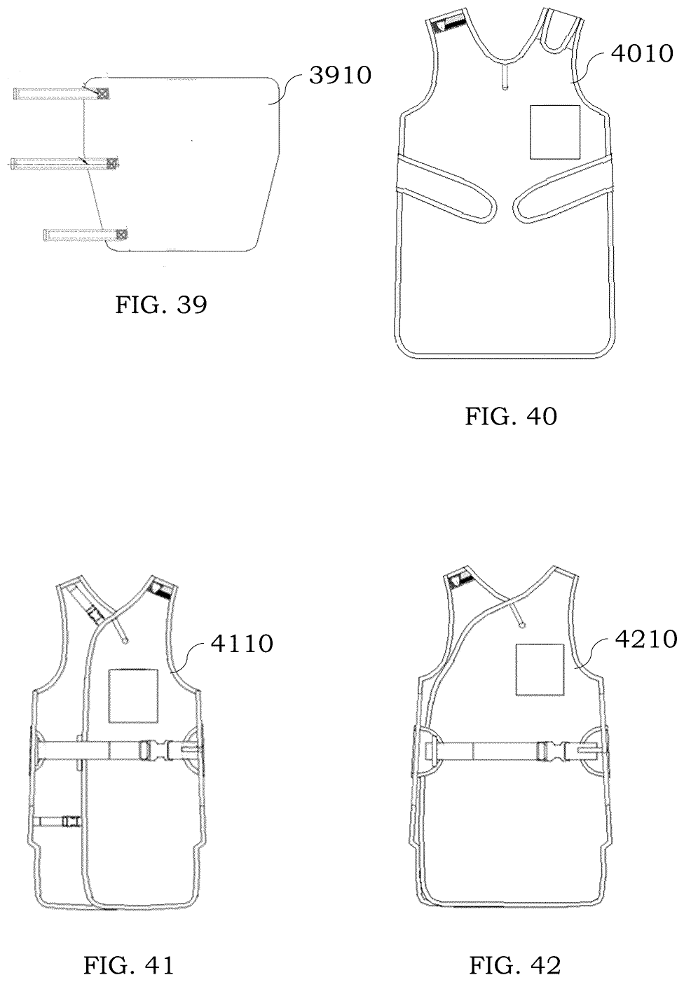

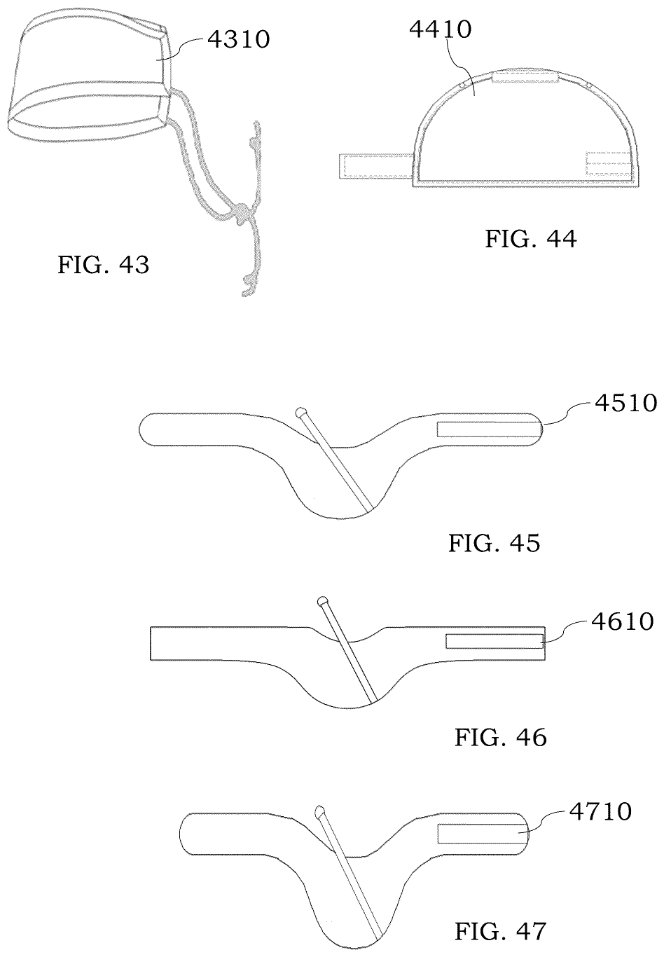

In certain embodiments, the protective compositions described herein can be present in an X-ray protective apron 3110, 3210 or 3310 as shown in FIGS. 31, 32 and 33, respectively. In other embodiments, the protective compositions described herein can be present in an X-ray protective vest 3410, 3510, 3610 or 3710 as shown in FIGS. 34, 35, 36 and 37, respectively. FIG. 38 shows illustrative X-ray protective skirt 3810. FIG. 39 shows an illustrative leg X-ray protective wrap 3910. FIGS. 40, 41 and 42 show illustrative X-ray protective body wraps 4010, 4110 and 4210, respectively. FIG. 43 shows a drawing of an illustrative X-ray protective cap 4310. FIG. 44 shows a drawing of an illustrative arm X-ray protective sleeve 4410. FIGS. 45, 46 and 47 show illustrative X-ray protective thyroid shields 4510, 4610 and 4710, respectively.

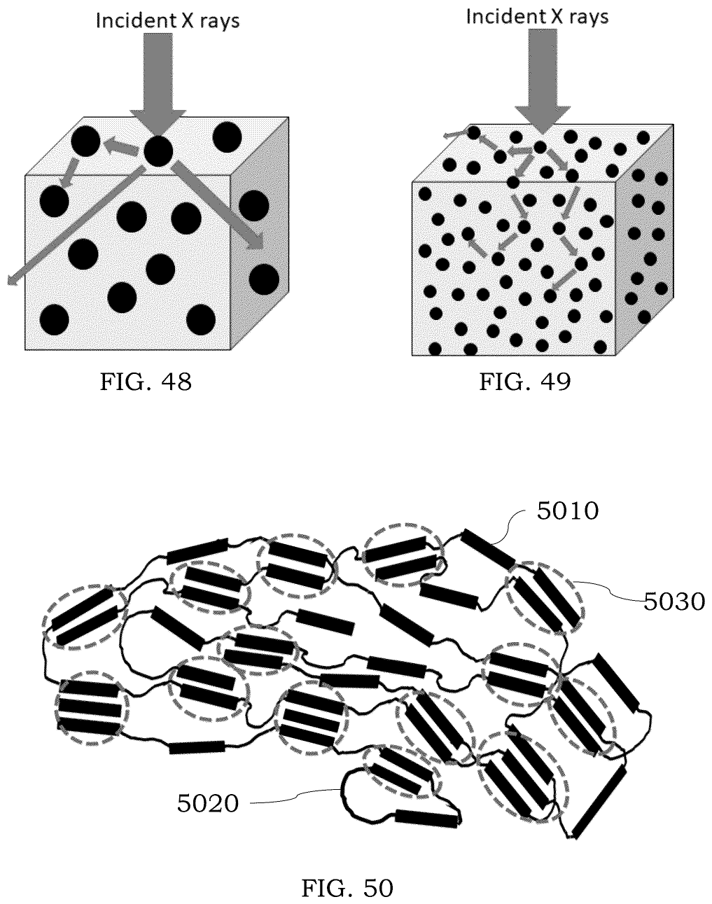

In certain embodiments, the compositions and articles described herein may comprise an elastomer in combination with micron size metal particles or nanosize metal particles or both. Illustrations are shown in FIGS. 48 and 49 where micron size particles in combination with an elastomer (FIG. 48) are shown as scattering incident X-rays 4820 into scattered X-rays 4820. In the elastomer with nanosize particle illustration (FIG. 49), the incident X-rays can be deflected into lower energy particles within the structure. While not shown, lead particles in combination with non-lead particles can be present and used to attenuate both primary X-rays and scattered X-rays.

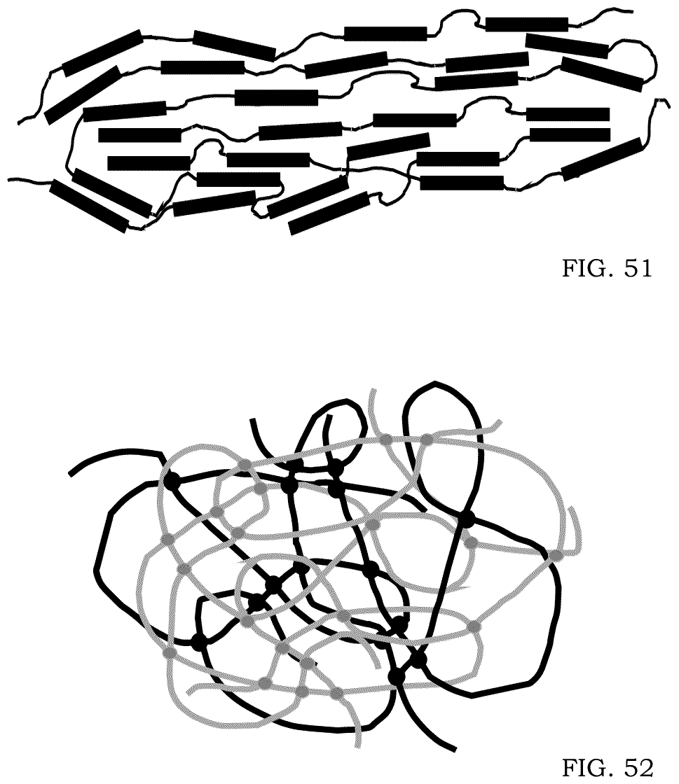

In other embodiments, the compositions and articles described herein may comprise an elastomeric composition that include different types of building blocks. For example and referring to FIG. 50, rigid building blocks 5010 and soft building blocks 5020 are shown. In some embodiments, the rigid segments 5010, when in close proximity, form crystallites 5030, yielding semi-crystalline final material. The metal particles in the composition are omitted in FIG. 50 for clarity. If desired, the matrix can be composed of rubbery soft segments and glassy or crystalline hard segments, showing a two-phase microstructure, in which the hard segments play a role in physical crosslinking and strengthening the structure of the polymer, while the soft segments give elasticity and ductility, thus acting as absorber for sudden and extended stresses, loads or impacts. Such two-phase microstructure can be provided by a block copolymer with rubbery (soft segments and hard crystalline segments). This two-phase microstructure can be also provided by graft copolymers or by blending hard semicrystalline polymers with elastomeric (rubbery) polymers.

In certain embodiments, the compositions and articles described herein may comprise liquid crystalline elastomers (LCEs) as shown in FIG. 51. LCEs exhibit excellent viscoelasticity, i.e. behaviors of both, elastic rubbery network and anisotropic properties of liquid crystals (LC), mechanical deformation, and actuation properties, among others. Some LCEs can deform and recover their shape when external stimuli are applied. LCE are preferred in embodiments where enhanced mechanical properties are needed in certain direction, such as curtains or other X ray shielding applications. In FIG. 51, the rectangles represent mesogen blocks and the lines represent flexible segments. In other embodiments the matrix material comprises at least one liquid crystalline elastomer (LCE). The LCE can be used alone or can be a blend of LCE and other materials described herein, e.g., thermoplastic materials, thermoset materials, etc.

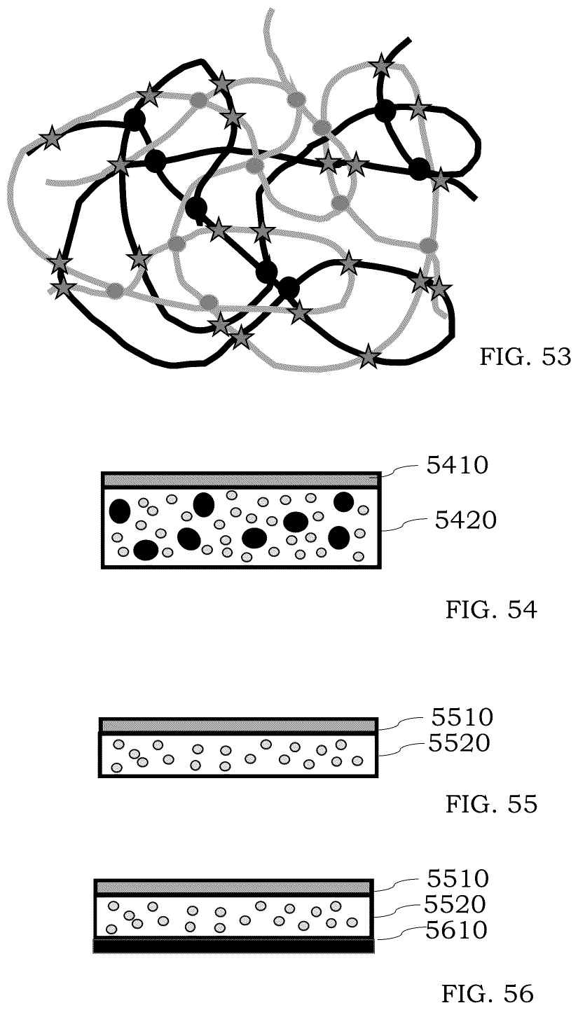

In some embodiments, the compositions and articles described herein may comprise an interpenetrating network (IPN). For example, the starting composition can be produced from a thermoset polymer (elastomer) and a thermoplastic polymer (elastomer), metal particles and additives. During processing, e.g. pelletizing and extrusion, the thermoset polymer undergoes cross-linking and the formed 3D network is around the thermoplastic polymer and captures the metal particles. This kind of s-IPN gives rise to both toughness and elasticity of the final material (FIG. 52). Another example is a combination of two thermoset elastomers (rubbers), which do not react with each other. Their cross-linking (e.g. vulcanization) occurs during the processing steps forming tough yet ductile IPN (FIG. 53).

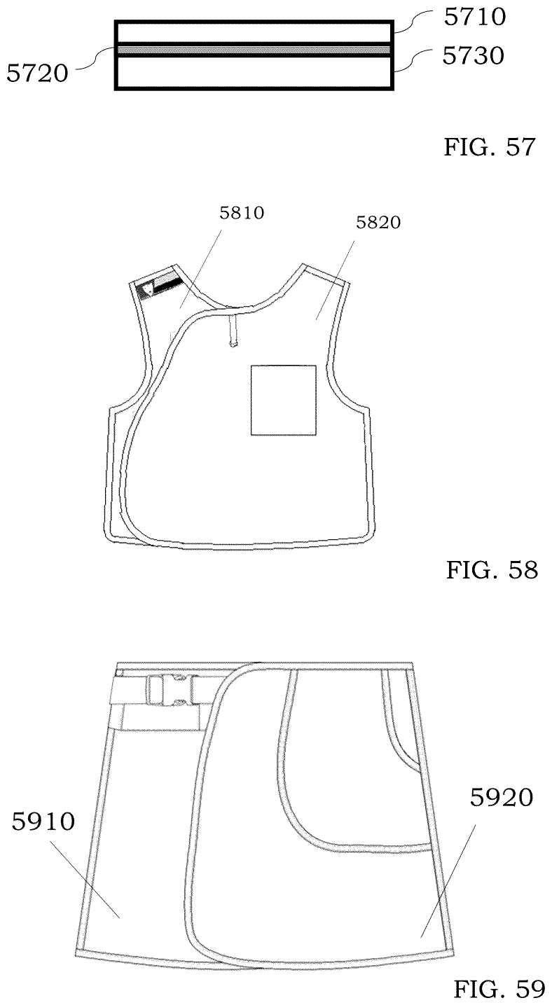

The exact process used to produce the compositions and articles described herein may vary. In general, the compositions can be produced by mixing, blending, compounding and/or reacting metals, metal particles, metal mixtures, etc. with the other components of the composition, e.g., with the polymers, elastomers, fillers, additives, etc. The materials can be pelletized, processed or otherwise formed into a desired shape, size, etc. prior to use to form a sheet, article, etc. For example, a radiation-protective sheet can be produced by extrusion, co-extrusion, calendaring, compression, molding, 3D printing, substrate coating, dipping, brushing, spraying, etc. the composition. In some examples, one metal, or two or more metals, can be co-extruded with the elastomers, polymers, etc. to form a single extruded sheet containing all the material. Individual layers can be extruded, coated or disposed on each other. For example and referring to FIGS. 54-58, a functional coating 5410, e.g., a superhydrophobic, antimicrobial, self-cleaning, etc., can be extruded or otherwise deposited onto an X-ray protection layer 5420 as shown in FIG. 54 Referring to FIG. 55, a thin metal coating 5510 can be deposited, e.g., extruded onto, sprayed into, or otherwise coated onto, a X-ray protection layer 5520. Referring to FIG. 56, an additional layer 5610 can be coated into another surface of the X-ray protection layer 5520. Referring to FIG. 57, a thin metal layer 5720 is shown as being present between elastomeric layers 5710, 5730. While not shown, the elastomeric layers 5710, 5730 can be configured as an X-ray radiation protection layer as described herein. The layers 5710, 5730 can be the same or can be different. Additional elastomeric layers can be added to the stack shown in FIG. 57 with an optional metal layer (or layer of other materials) present between the elastomeric layers. The resulting sheet or article can be post-processed to provide a desired shape, size, etc. For example, additional layer or materials can be added to the radiation protective sheet by way of 3D printing, spraying, coating, cross-linking, lamination, photo-, e-beam- or thermal-polymerization, etc. The final article can be cut to size and/or shape to provide a suitable article for use in X-ray protection.

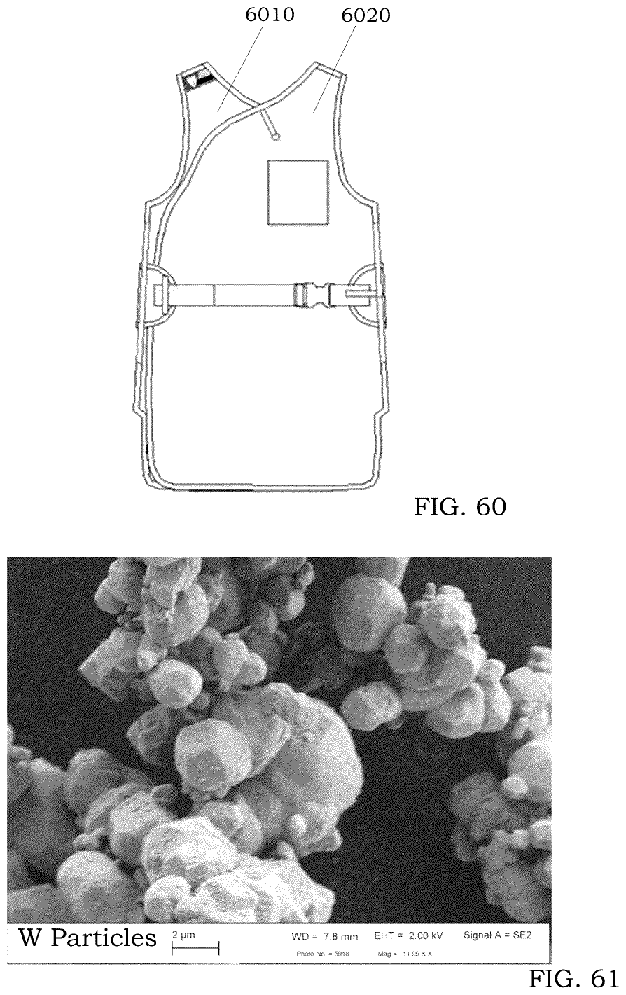

In certain embodiments, articles that include the protective compositions described herein may comprise different materials at different areas. Referring to FIG. 58, a radiation vest is shown that has areas 5810 and 5820. For example, area 5810 may have a higher absorption metal and area 5820 may have a lower absorption metal. The back of the vest may have a mix of both metals to enhance absorption. Referring to FIG. 59, area 5910 of an X-ray shielding skirt may have a high absorption metal and area 5920 may have a lower absorption metal. The back surface can include both materials to increase shielding. Referring to FIG. 60, an X-ray shielding apron can include areas 6010, 6020. Area 6010 may have a high absorption metal and area 6020 may have a lower absorption metal. The back surface can include both materials to increase shielding.

Certain specific examples are described to illustrate further some of the novel and inventive aspects described herein. In the following examples, certain micron- and sub-micron size radiation protective metal particles were used in the preparation of radiation-protective materials.

### Example 1

Scanning electron microscope (SEM) images of some of the radiation-attenuating particles, such as W, Sb, Bi, Bi₂O₃, Sn and BaSO₄, are shown in FIGS. 61-66. Tungsten (W) is a metal with high density but good radiation attenuating properties. W particles with sizes of less than 5 μm are particularly useful in the compositions described herein (FIG. 61) to provide the broad range protection, especially from scatter X-rays without contributing a lot to the weight of the protective garment. Therefore, W particles are used in small fraction of the protective material composition, ranging from 5% wt to about 20% wt, or if used in multi-layers structure, it is preferably used as thin layers comprising the W particles.

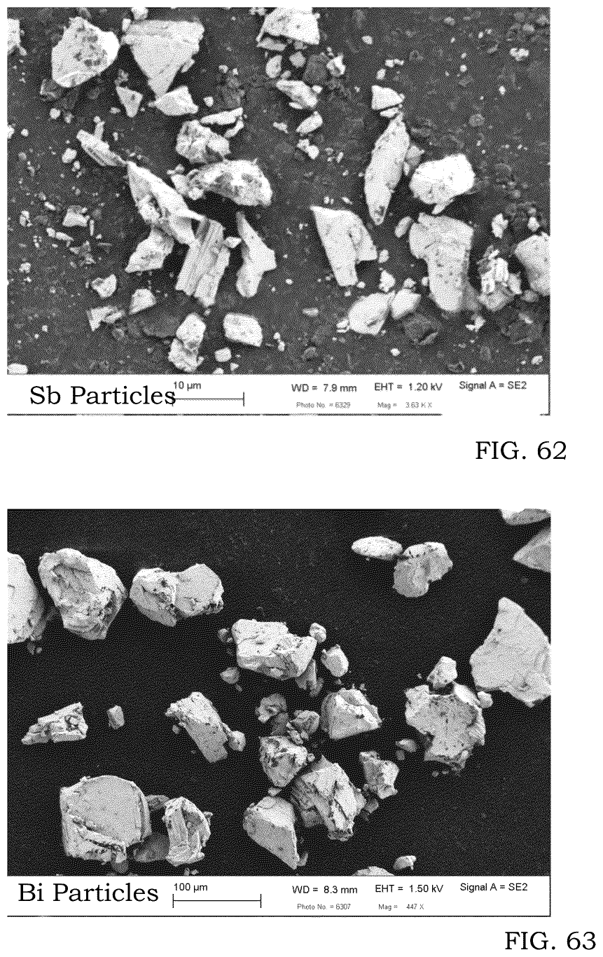

Sb is a radiation-protective metal usually used to provide shielding from medium and energy X-rays, where the particle size is not a critical parameter. Therefore, Sb particles with sizes in the range of 5-50 μm (FIG. 62) are useful in the compositions provided herein in combination with other metal particles.

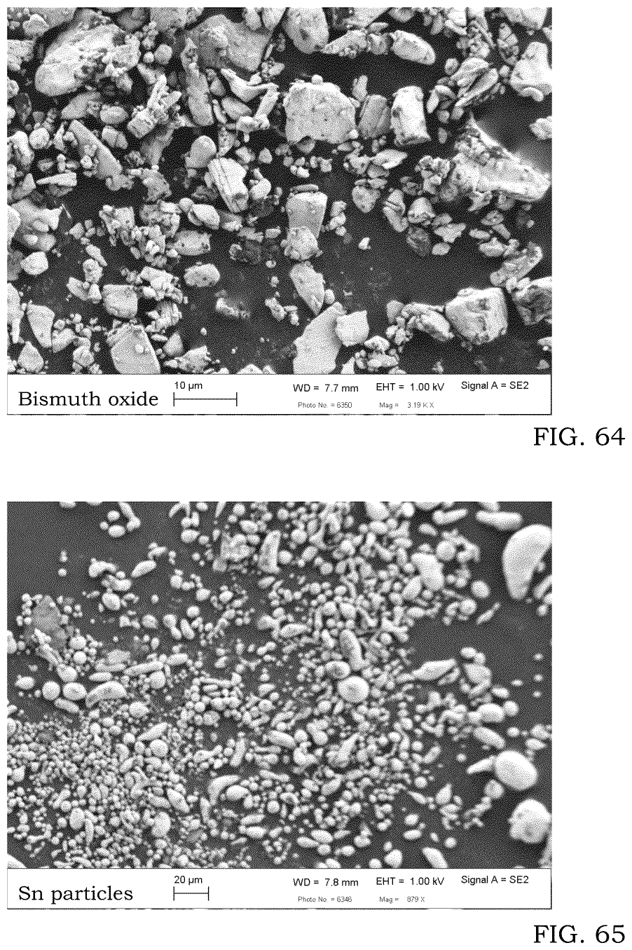

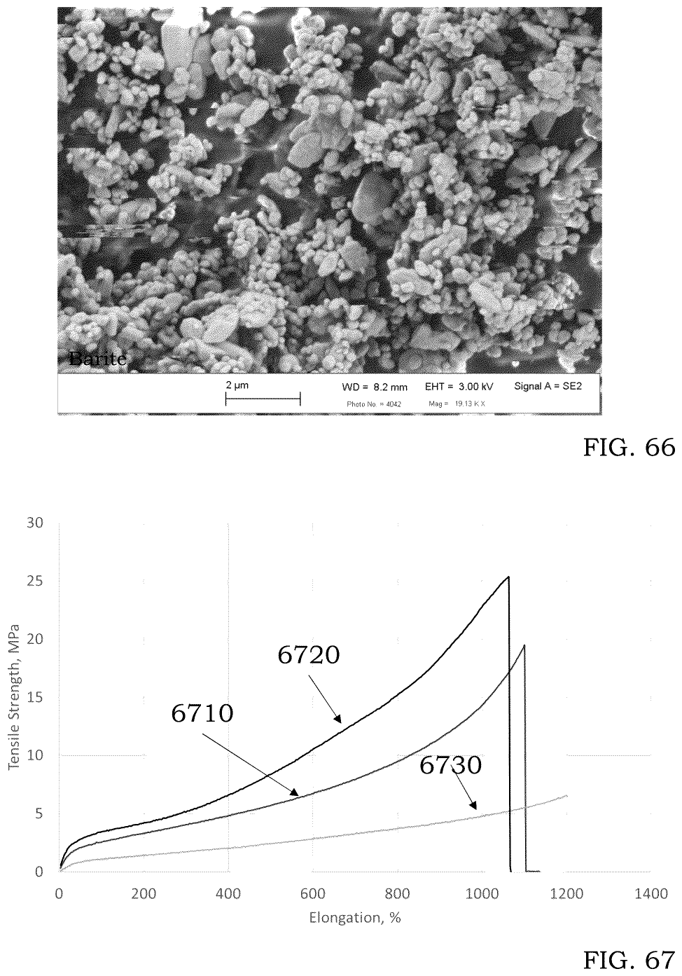

Bi has a long mean free path and its shielding characteristics are very close to those of Pb. For this reason, Bi is a desirable material to include in the X-ray shielding materials described herein. Targeting the low-energy photons as a result of the primary X-ray beam scattering, micron-size and nano-sized particles of Bi are used (FIGS. 63 and 64), which resulted in a considerable improvement in the radiation-shielding performance. Sn and barite (BaSO₄) can also be used in the compositions and articles descried herein (FIGS. 65 and 66) as metals combined with other metals to provide efficient and broad range radiation protection.

### Example 2

Various materials were tested for their ability to be used as elastomeric matrices in X-ray protective compositions. The tested materials were vinyl chloride (plastisol), ethylene-vinyl hexyl copolymer and ethylene-vinyl-acetate copolymer (both 28% vinyl-acetate groups and 40% vinyl-acetate groups).

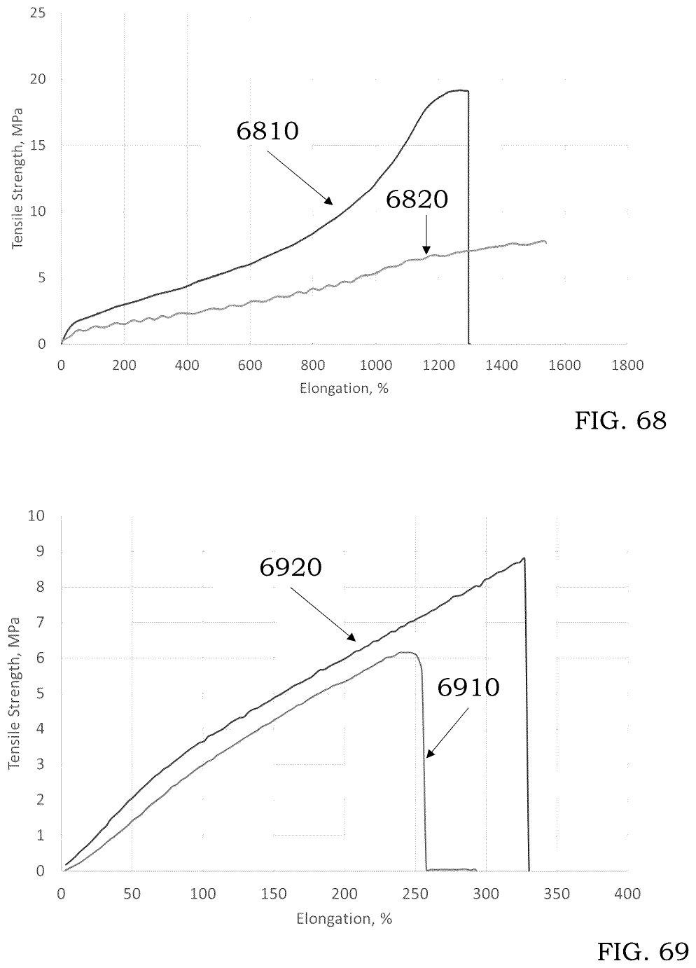

Referring to FIG. 67, mechanical properties of ethylene-vinyl hexyl copolymer (arrow 6710) and ethylene-vinyl-acetate copolymer (28% vinyl-acetate groups—arrow 6720) and ethylene-vinyl-acetate copolymer (40% vinyl-acetate groups—arrow 6730) are shown. The polymers were stretched at 500 mm/minute. Ethylene-vinyl-acetate copolymer (40% vinyl-acetate groups) did not break.

Referring to FIG. 68, mechanical properties of ethylene-vinyl hexyl copolymer (arrow 6810) and ethylene-vinyl-acetate copolymer (40% vinyl-acetate groups—arrow 6830) are shown. The polymers were stretched at 50 mm/minute. Ethylene-vinyl-acetate copolymer (40% vinyl-acetate groups) did not break.

Referring to FIG. 69, polyvinyl chloride was stretched at 500 mm/minute at two temperatures −160 deg. Celsius (arrow 6910) and 180 deg. Celsius (arrow 6920).

The results were consistent with these materials being suitable for use as matrices in the compositions described herein.

### Example 3

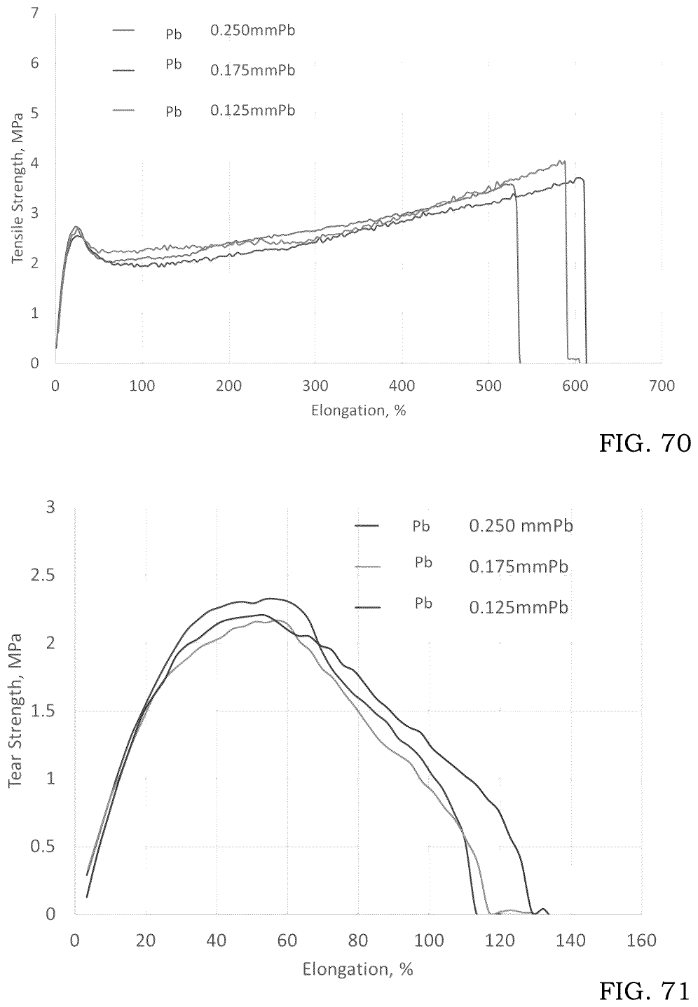

Lead containing samples in a polyolefin elastomers were prepared and tested for their ability to attenuate X-rays. The tensile stress-strain curves are shown in FIG. 70. Tear strength measurements are shown in FIG. 71.

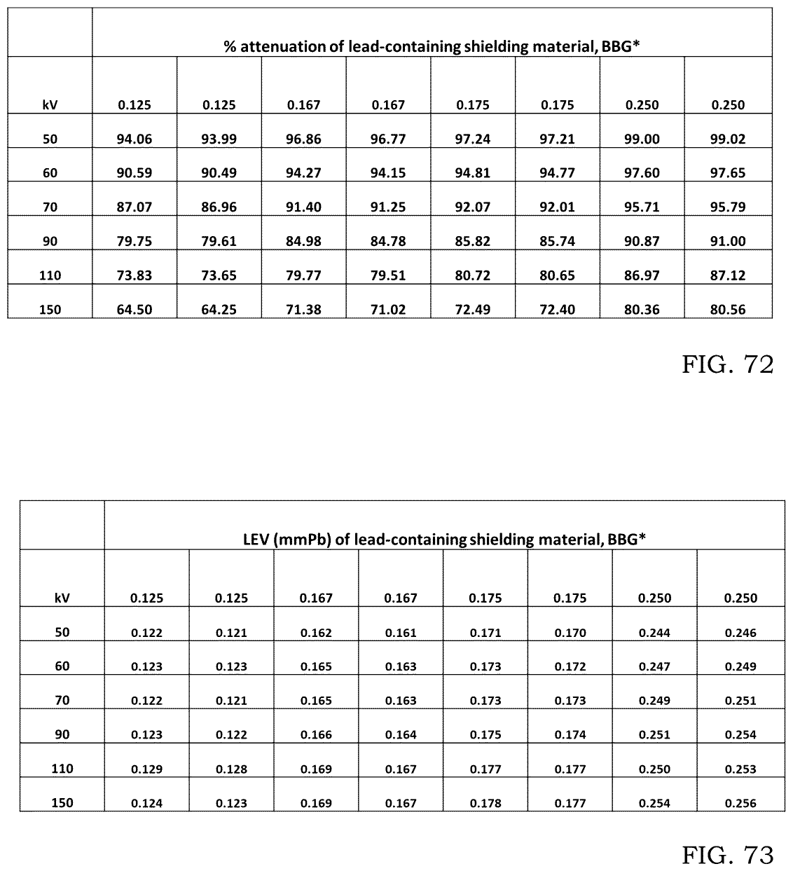

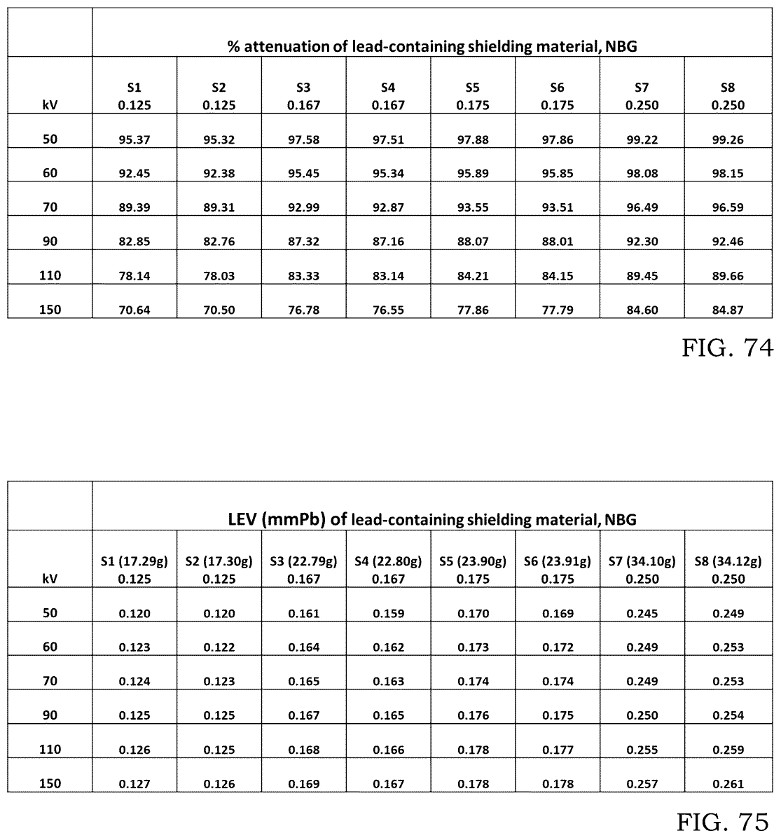

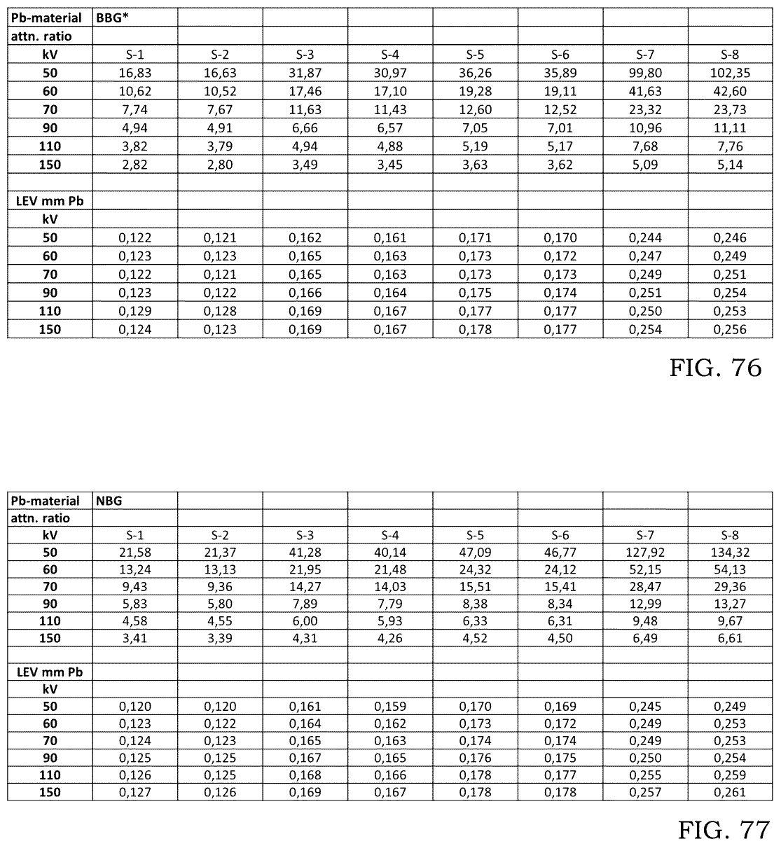

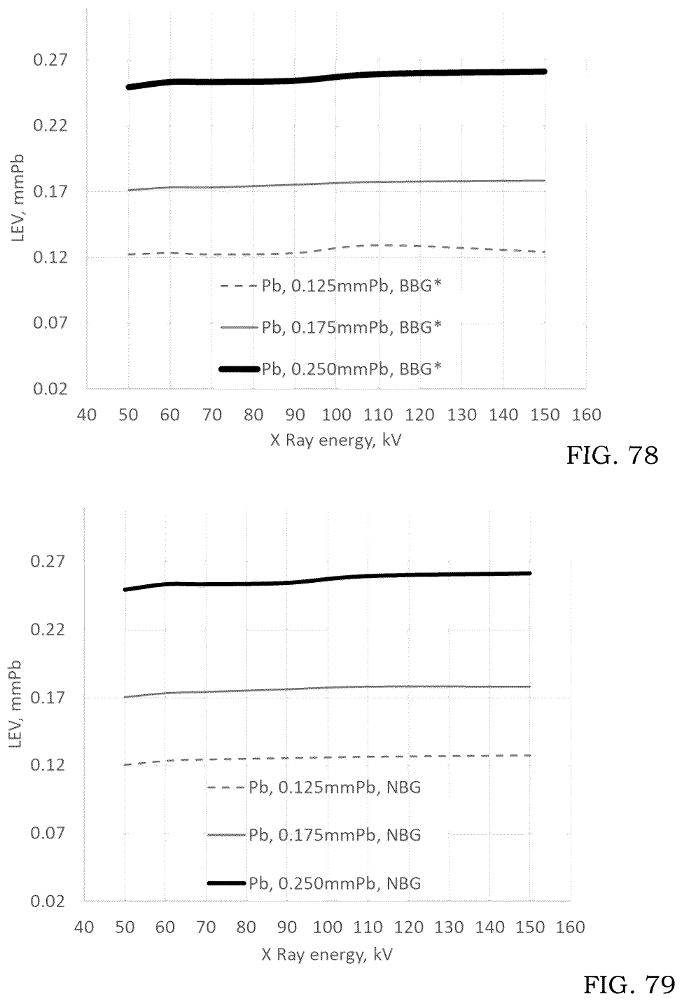

Percentage attenuation values were calculated as different source voltages according to the IEC/EN 61331-1:2014 protocol. The results are shown in FIG. 72 for BBG* measurements. LEV calculations based on the BBG* measurements are shown in FIG. 73. The results are shown in FIG. 74 for NBG measurements. LEV calculations based on the NBG measurements are shown in FIG. 75. Attenuation ratios for the BBG* and NBG measurements are shown in FIG. 76 and FIG. 77, respectively. LEV values were calculated from the attenuation data measured in the BBG* mode (FIG. 78) and the NBG mode (FIG. 79). The results were consistent with the composition being able to provide attenuation of the X-rays in a generally linear manner over the range of 50-150 kV source voltage.

### Example 4

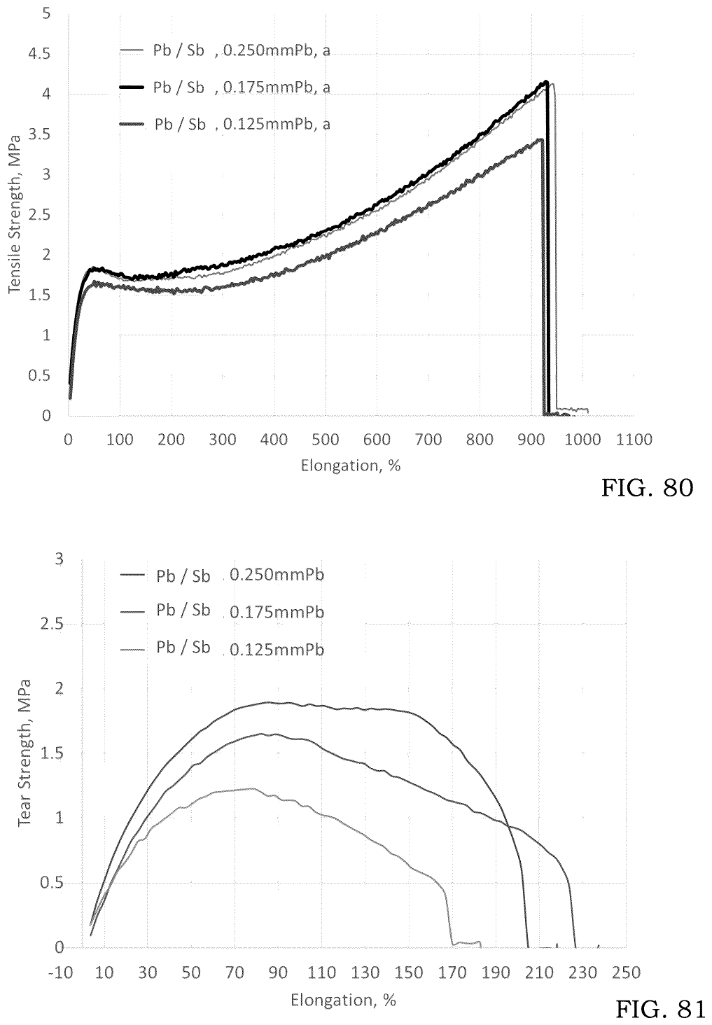

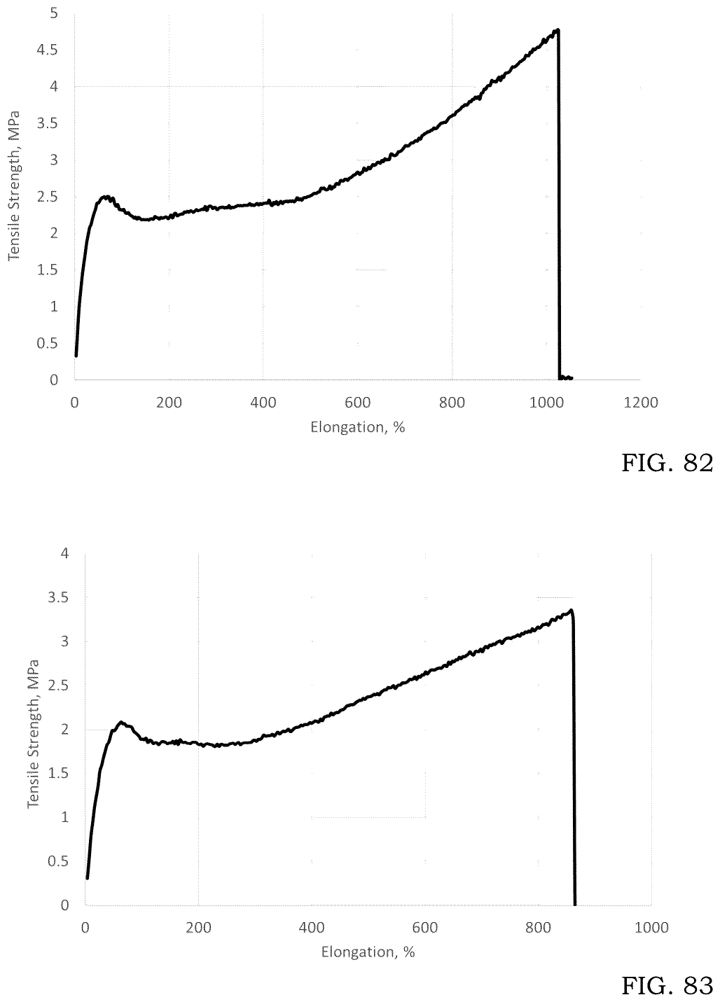

Pb—Sb and Pb—Sb—Ba containing samples in a polyolefin elastomers were prepared and tested for their ability to attenuate X-rays. The tensile stress-strain curves are shown in FIG. 80 for the Pb—Sb compositions. Tear strength measurements are shown in FIG. 81 for the Pb—Sb compositions. The tensile stress-strain curves are shown in FIG. 82 for the Pb—Sb—Ba compositions. In the Pb—Sb—Ba compositions, lead was present in an higher amount that Sb or Ba. The amount of Sb was higher than the amount of Ba. The exact amounts of each metal can vary. The tensile stress-strain curves are shown in FIG. 83 for the Pb—Sb—Ba—W compositions. Pb was present in higher amounts than the other three metals.

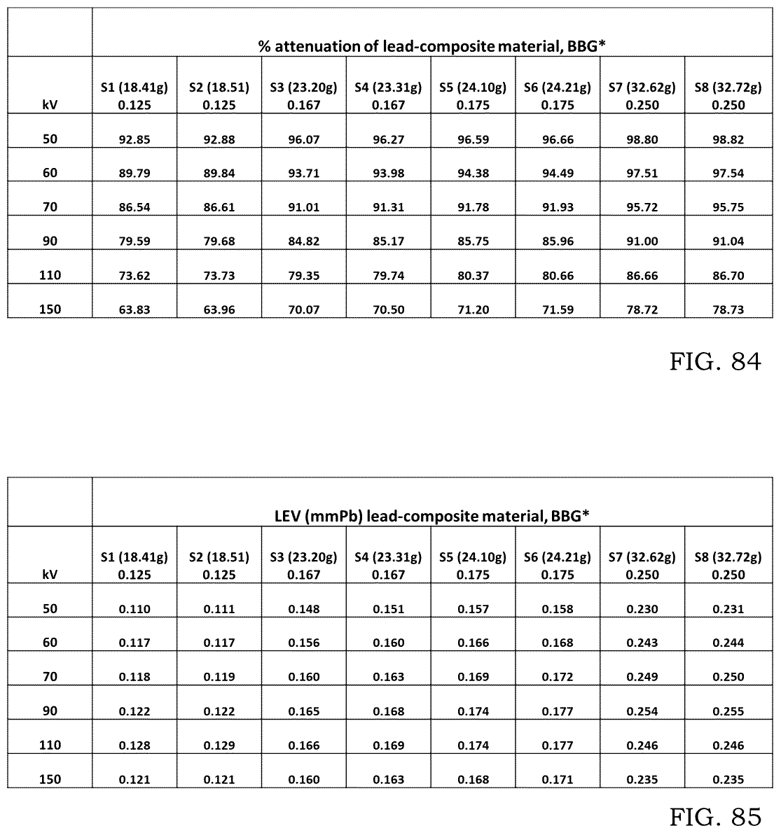

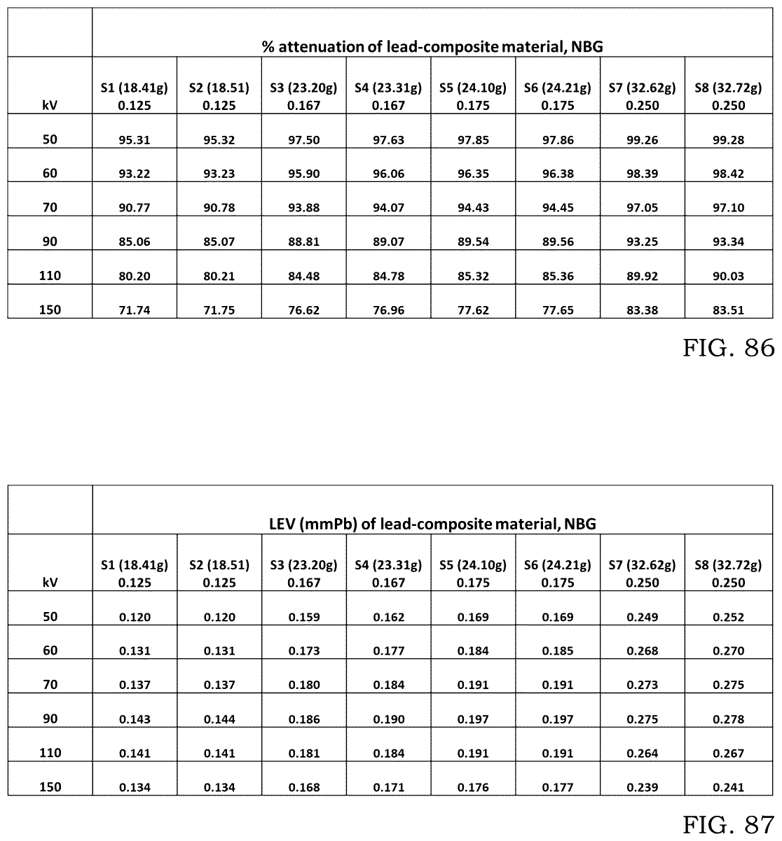

FIG. 84 shows the percentage attenuation of the Pb—Sb compositions using BBG* measurements. LEV values were calculated from the attenuation data measured in the BBG* mode for the Pb—Sb compositions (FIG. 85). FIG. 86 shows the percentage attenuation of the Pb—Sb compositions using NBG measurements. LEV values were calculated from the attenuation data measured in the NBG mode for the Pb—Sb compositions (FIG. 87).

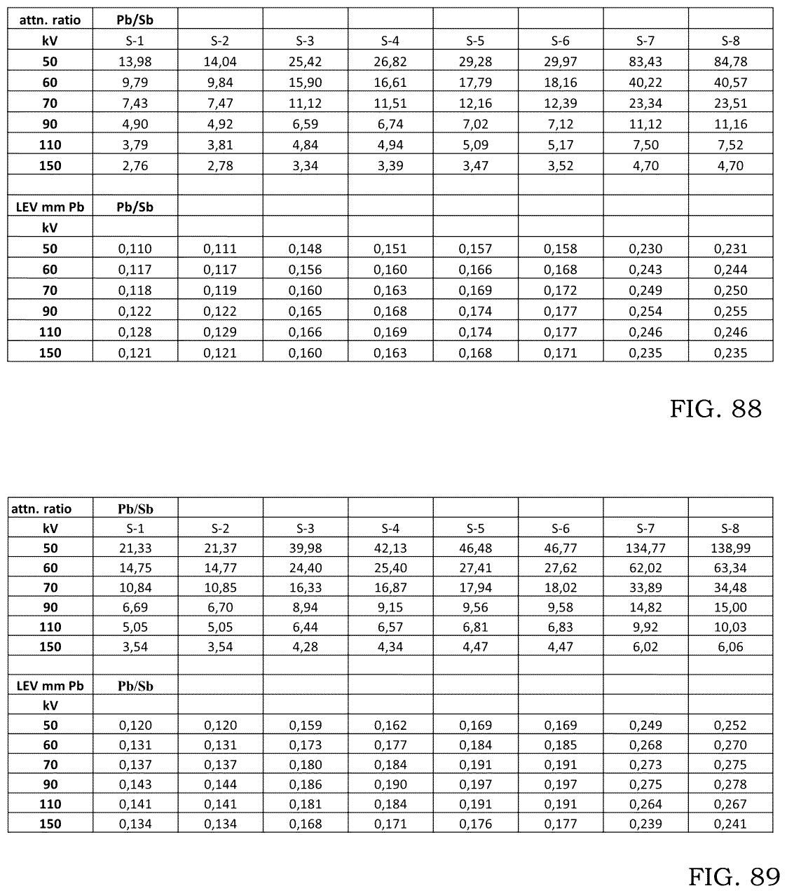

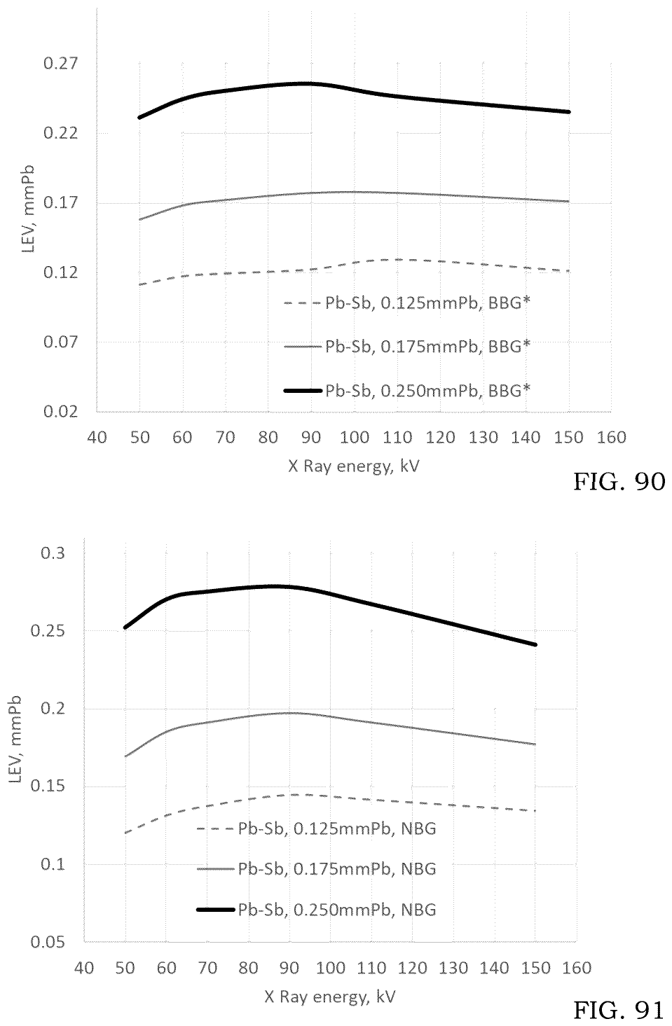

Attenuation ratios are shown in FIG. 88 (BBG* mode) and FIG. 89 (NBG mode) for the Pb—Sb compositions. LEV values were calculated from the attenuation data measured in the BBG* mode (FIG. 90) and the NBG mode (FIG. 91). The results were consistent with the composition being able to provide attenuation of the X-rays in a generally linear manner over the range of 50-150 kV source voltage.

### Example 5

Several compositions were tested that included both recycled and virgin materials. The compositions included Pb, Sb, Ba and W with differing amounts of recycled materials including 0% by weight recycled material, 30% by weight recycled material and 60% by weight recycled material. The recycled material included the same material as the virgin material but has been previously used, was grinded and then combined with the virgin material. B3E refers to the virgin material.

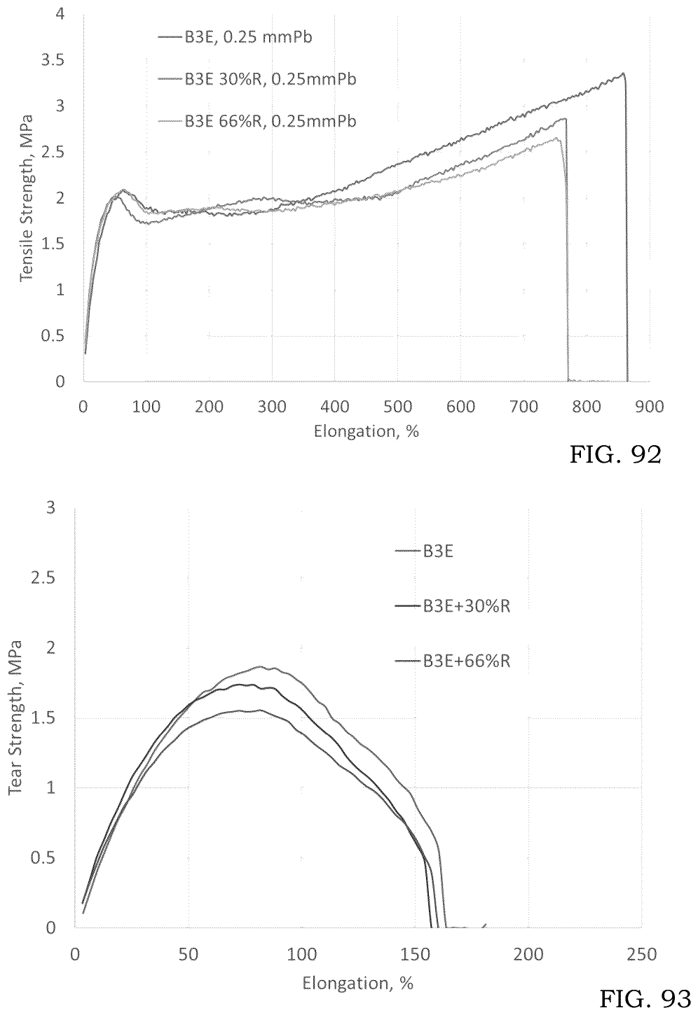

FIG. 92 shows the tensile strength properties with different amounts of recycled material. The curves show similar properties. The samples were all with LEV of 0.250 mmPb and stretched at 500 mm/min. Tear strengths were also measured for the different compositions (FIG. 93). The samples were all with LEV of 0.250 mmPb and stretched at 500 mm/min.

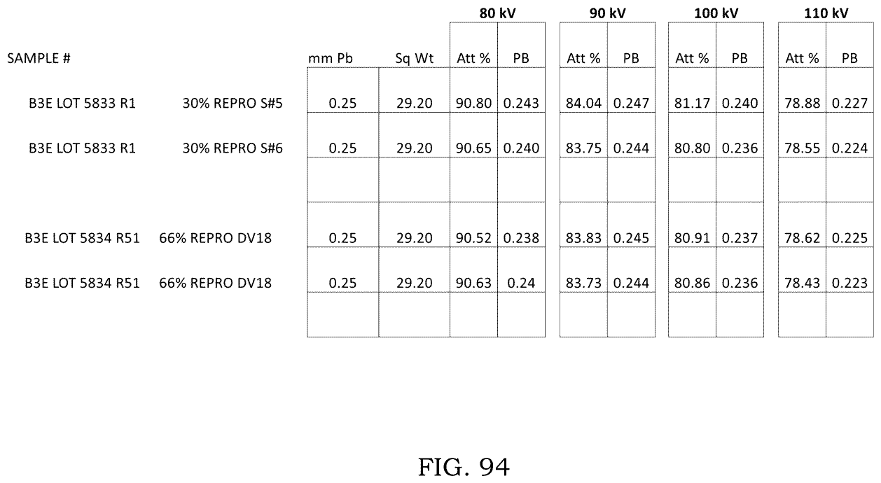

FIG. 94 shows a table of attenuation measurements (NBG) for different amounts of recycled content at different source voltages. The attenuation values were similar at different recycled content for a particular source voltage value.

### Example 6

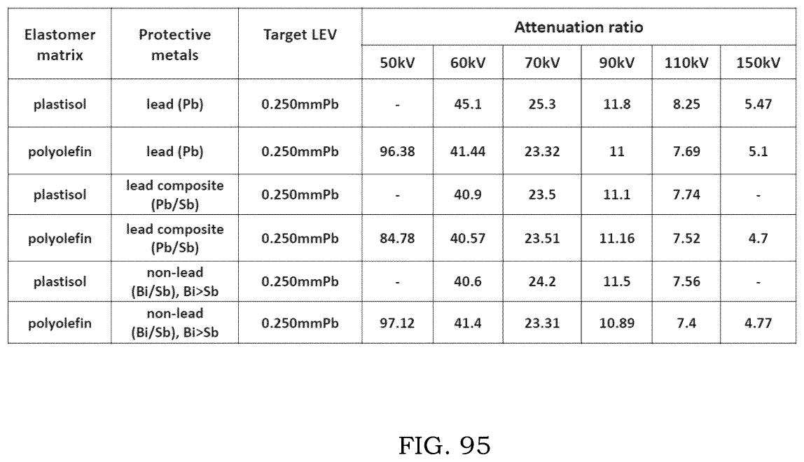

Attenuation ratios were measured for several lead and non-lead compositions with different elastomer matrices as shown in FIG. 95. The target LEV was 0.250 mmPb for each measurement. The Pb—Sb compositions provide similar attenuation ratios as lead based compositions.

In summary, the disclosed lead-metal-filled elastomeric compositions and the protective sheet materials made of such compositions in a single-, bi- and multi-layer structures and hybrid structures, have the ability to effectively protect against a broad range of X-ray energies including primary X-rays and scattered X-rays. The protective garments made of such material sheets are light-weight and flexible with improved resistance to cracks, abrasion and durability.

When introducing elements of the aspects, embodiments and examples disclosed herein, the articles “a,” “an,” “the” and “said” are intended to mean that there are one or more of the elements. The terms “comprising,” “including” and “having” are intended to be open-ended and mean that there may be additional elements other than the listed elements. It will be recognized by the person of ordinary skill in the art, given the benefit of this disclosure, that various components of the examples can be interchanged or substituted with various components in other examples.

Although certain aspects, examples and embodiments have been described above, it will be recognized by the person of ordinary skill in the art, given the benefit of this disclosure, that additions, substitutions, modifications, and alterations of the disclosed illustrative aspects, examples and embodiments are possible.

1. A lead containing radiation-shielding metal-filled elastomeric composition comprising:
lead and at least one metal selected from heavy metals known to have shielding capability against ionizing radiation and having atomic (Z) number of at least 50;
a matrix comprising at least one polymer, polymer blend or co-polymer selected from the group consisting of thermoplastic polymers, thermoset polymers, elastomeric polymers, visco-elastic polymers, and combinations thereof,
wherein the lead and the at least one metal are together in the range from 50 wt % to about 90 wt % based on the weight of the lead containing radiation-shielding metal-filled elastomeric composition, and wherein the lead and the at least one metal are arranged in the matrix to provide protection from both primary X-rays and scatter X-rays.

2. The composition of claim 1, wherein the at least one polymer, polymer blend or co-polymer is polyvinyl chloride (PVC) elastomer, and wherein the composition further comprises a plasticizer.

3. The composition of claim 1, where the at least one polymer, polymer blend or co-polymer is selected from the group consisting of a polyolefin elastomer, a natural rubber, a synthetic rubber, a urethane-type elastomer, a silicone-type elastomer, a vinyl acetate polymer, a vinyl chloride polymer, an ethylene-vinyl hexyl copolymer, an ethylene-vinyl acetate copolymer, a blend of at least two different types of polymers, and a co-polymer comprising at least two different monomer repeating units.

4. The composition of claim 1, where the metal is a mixture of at least two different heavy metals each with Z-numbers of at least 50.

5. The composition of claim 1, where the metals used are in a form of metal alloy or used in its pure elemental form or a metal carbonate, a metal sulfate, a metal oxide, or a combination of different metal forms.

6. The composition of claim 1, where the at least one polymer, polymer blend or co-polymer is present in a fiber.

7. The composition of claim 6, wherein the heavy metal is coated into the fiber.

8. The composition of claim 7, wherein the fiber comprises one or more of polyethylene (PE), polypropylene (PP), polyester terephthalate (PET), polyethylene naphthalate (PEN), nylon, polyacrylonitrile (PAN), polyamide, polycarbonate (PC), aramid, or combinations thereof.

9. The composition of claim 1, wherein the heavy metal is distributed in the at least one polymer, polymer blend or co-polymer in a substantially uniform distribution or in a gradient distribution.

10. The composition of claim 1, wherein the metal is a mixture of two different heavy metals and wherein one of the heavy metals of the mixture is present in a non-uniform distribution in the polymer, polymer blend or co-polymer.

11. The composition of claim 1, wherein the heavy metals include Bi in combination with one or more of Sb, W, Sn or Ba, wherein the Bi is present in an amount that exceeds the amount of each of the Sb, W, Sn or Ba.

12. The composition of claim 1, wherein the heavy metals include Sb in combination with one or more of Bi, W, Sn or Ba, wherein the Sb is present in an amount that exceeds an amount of each of the Bi, W, Sn or Ba.

13. The composition of claim 1, wherein the heavy metals include W in combination with one or more of Bi, Sb, Sn or Ba, wherein the W is present in an amount that exceeds an amount of each of the Bi, Sb, Sn or Ba.

14. The composition of claim 1, wherein the heavy metals include Ba in combination with one or more of Bi, Sb, W or Sn, wherein the Ba is present in an amount that exceeds an amount of each of the Bi, Sb, W or Sn.

15. The composition of claim 1, wherein the at least one metal is present in the composition as particles, and wherein the particles have different sizes including micron size particles and nanosize particles.

16. A radiation-shielding material sheet comprising the lead containing radiation-shielding metal-filled elastomeric composition of claim 1.

17. The radiation-shielding material sheet of claim 16, wherein the sheet comprises a first layer and a second layer, and wherein the lead containing radiation-shielding metal-filled elastomeric composition is present in the first layer.

18. The radiation-shielding material sheet of claim 16, where the sheet is a single layer comprising the lead containing radiation-shielding metal-filled elastomeric composition.

19. The radiation-shielding material sheet of claim 16, where the sheet comprises a first layer and a second layer, wherein the lead containing radiation-shielding metal-filled elastomeric composition is present in the first layer, and wherein the second layer comprises an additional lead containing radiation-shielding metal-filled elastomeric composition comprising at least one meta selected from heavy metals known to have shielding capability against ionizing radiation, and having atomic (Z) number of at least 50, at least one polymer, polymer blend or co-polymer selected from the group consisting of elastomers, visco-elastic polymers, and additives, such as plasticizers, wetting agents, flow modifiers and others, wherein the at least one metal in the additional lead containing radiation-shielding metal-filled elastomeric composition is in the range from 50 wt % to about 90 wt % based on the weight of the additional lead containing radiation-shielding metal-filled elastomeric composition, and wherein the at least one metal is arranged in the matrix of the additional lead containing radiation-shielding metal-filled elastomeric composition to provide protection from both primary X-rays and scatter X-rays.

20. The radiation material sheet of claim 16, wherein the sheet comprises a first layer and a second layer, and wherein the second layer comprises a fabric.

21-24. (canceled)
